(19) United States
(12) Patent Application Publication
(10) Pub. No.: US 2018/0082012 A1
(43) Pub. Date: Mar. 22, 2018

(54) Method and device for determining fraction of cell-free nucleic acids in biological sample and use thereof

(71) Applicant: BGI GENOMICS CO., LTD., Shenzhen (CN)

(72) Inventors: Fuman Jiang, Shenzhen (CN); Yuying Yuan, Shenzhen (CN); Wei Wang, Shenzhen (CN); Ye Yin, Shenzhen (CN)

(21) Appl. No.: 15/329,148

(22) PCT Filed: Jul. 24, 2015
(86) PCT No.: PCT/CN2015/085109

§ 371 Date: Jan. 25, 2017

(30) Foreign Application Priority Data
Jul. 25, 2014 (CN) 201410359726.4

(51) Int. Cl. G06F 19/16; G06F 19/22; G06F 19/18; C12Q 1/68

(52) U.S. Cl. CPC G06F 19/16; C12Q 1/6869; G06F 19/18; G06F 19/22

(57) ABSTRACT

Provided in the present disclosure are a method and a device for determining a fraction of cell free nucleic acids in a biological sample and use thereof, wherein the method comprises: (1) sequencing nucleic acids of a biological sample having free nucleic acids, in order to obtain sequencing results for a plurality of sequencing data; (2) based on the sequencing results, determining the number of nucleic acid molecules with a length falling within a preset range in the sample; and (3) based on the number of nucleic acid molecules with a length falling within the preset range, determining the ratio of free nucleic acids in the biological sample.

### FIELD

The present disclosure relates to the field of biotechnology, in particular to a method and a device for determining a fraction of cell-free nucleic acids in a biological sample and their uses.

### BACKGROUND

Since 1977, researchers have successively found cancer-derived DNA in peripheral blood of patients with tumor, also confirmed the presence of cff-DNA in plasma from a pregnant woman. Detection or estimation of cancer-derived DNA in peripheral blood of patients with tumor and cell-free fetal DNA fraction in the plasma from the pregnant woman, i.e. determination of a fraction of cell-free nucleic acids from a predetermined source in a biological sample, is of great significance.

However, the current method for determining the fraction of cell-free nucleic acids in the biological sample remains to be unproved.

### SUMMARY

Embodiments of the present disclosure seek to solve at least one of the problems existing in the related art to at least some extent. For this, an object of the present disclosure is to provide a method capable of accurately and efficiently determining a fraction of cell-free nucleic acids from a predetermined source in a biological sample.

It should be noted that the technical solutions of the present disclosure are achieved through the following discoveries.

At present, there are two main directions for estimating the fraction of cell-free fetal DNA fraction in peripheral blood: 1) taking advantage of different responses of maternal DNA fragments and cell-free fetal DNA fragments in mononuclear cells from maternal peripheral blood to methylation of specific markers; 2) selecting a plurality of representative SNPs sites based on differences among single nucleotide polymorphism (SNPs) sites. Both methods have a certain limitation: Method 1) requires a large amount of plasma, and method 2) requires probe capture and high sequencing depth, or needs to obtain parental information. However, no report has so far been published that cell-free fetal DNA fraction is estimated under whole genome sequencing at low coverage depth. Studies have shown that cell-free fetal DNA fragments in maternal blood circulation, majority of which are shorter than 313 bp, axe generally shorter than cell-free maternal DNA fragments. Under this inspiration, the inventors have invented a procedure for estimating cell-free fetal DNA fraction based on sequencing the plasma from the pregnant woman, and which method has wide applications and may be applied to cell-free DNA from different sources. For example, this method may also be used to estimate a cancer-derived DNA fraction in peripheral blood of a patient with tumor.

In a first aspect, the present disclosure provides a method for determining a fraction of cell-free nucleic acids from a predetermined source in a biological sample. In embodiments of the present disclosure, the method includes: performing sequencing on cell-free nucleic acids contained in the biological sample, so as to obtain a sequencing result consisting of a plurality of sequencing data; determining the number of the cell-free nucleic acids in a length falling into a predetermined range in the biological sample based on the sequencing result; and determining the fraction of the cell-free nucleic acids from the predetermined source in the biological sample based on the number of the cell-free nucleic acids in the length falling into the predetermined range. The inventors have surprisingly found that, the fraction of the cell-free nucleic acids from the predetermined source in the biological sample, especially the fraction of the cell-free fetal nucleic acids in a peripheral blood sample obtained from a pregnant woman, or the fraction of cell-free tumor derived nucleic acids in a peripheral blood sample obtained from a subject suffering from tumor, suspected to suffer from tumor or subjected to tumor screening can be accurately and efficiently determined by the method of the present disclosure.

In embodiments of the present disclosure, the biological sample is a peripheral blood sample.

In embodiments of the present disclosure, the cell-free nucleic acid from the predetermined source is selected from one of the followings: cell-free fetal nucleic acids or maternal cell-free nucleic acids in a peripheral blood sample obtained from a pregnant woman, or cell-free tumor derived nucleic acids or cell-free non-tumor derived nucleic acids in a peripheral blood sample obtained from a subject suffering from tumor, suspected to suffer from tumor or subjected to tumor screening. Therefore, the fraction of the cell-free fetal nucleic acids in a peripheral blood sample obtained from a pregnant woman, or the fraction of cell-free tumor derived nucleic acids in a peripheral blood sample obtained from a subject suffering from tumor can be easily determined.

In some specific embodiments of the present disclosure, the cell-free nucleic acids are DNA.

In embodiments of the present disclosure, the sequencing result includes lengthes of the cell-free nucleic acids.

In embodiments of the present disclosure, the cell-free nucleic acids in the biological sample are sequenced by paired-end sequencing, single-end sequencing or single molecule sequencing. Therefore, lengthes of the cell-free nucleic acids may be obtained easily, which is conducive to subsequent steps.

In embodiments of the present disclosure, the cell-free nucleic acids are DNA.

In embodiments of the present disclosure, determining the number of the cell-free nucleic acids in the length falling into the predetermined range in the biological sample based on the sequencing result further includes: aligning the sequencing result to a reference genome, so as to construct a dataset consisting of a plurality of uniquely mapped reads, where each read in the dataset can be maped to a position of the reference genome only; determining a length of the cell-free nucleic acid corresponding to each uniquely mapped read in the dataset; and determining the number of the cell-free nucleic acids in the length falling into the predetermined range. Therefore, the number of the cell-free nucleic acids in the length falling into the predetermined range in the biological sample can be determined easily, which gives rise to an accurate and reliable result and good reproducibility.

In embodiments of the present disclosure, determining a length of the cell-free nucleic acid corresponding to each uniquely mapped read in the dataset further includes: determining the length of each read uniquely mapped to the reference genome as the length of the cell-free nucleic acid corresponding to the read. Therefore, the length of the cell-free nucleic acid corresponding to each uniquely-aligned read in the dataset can be determined accurately.

In embodiments of the present disclosure, in the case that the cell-free nucleic acids in the biological sample are sequenced by the paired-end sequencing, determining a length of the cell-free nucleic acid corresponding to each uniquely-mapped read in the dataset includes: determining a position, corresponding to the reference genome, of 5′-end of the cell-free nucleic acid, based on sequencing data at one end of each uniquely-mapped read obtained in the paired-end sequencing; determining a position, corresponding to the reference genome, of 3′-end of the cell-free nucleic acid, based on sequencing data at the other end of same uniquely-mapped read obtained in the paired-end sequencing; and determining the length of the cell-free nucleic acid based on the position of 5′-end of the cell-free nucleic acid and the position of 3′-end of the cell-free nucleic acid. Therefore, the length of the cell-free nucleic acid corresponding to each uniquely-mapped read in the dataset can be determined accurately.

In embodiments of the present disclosure, the predetermined range is determined based on a plurality of control samples, in each of which the fraction of the cell-free nucleic acids from the predetermined source is known. Therefore, the predetermined range can be determined with an accurate and reliable result.

In embodiments of the present disclosure, the predetermined range is determined based on at least 20 control samples.

In embodiments of the present disclosure, the predetermined range is determined by following steps: (a) determining lengths of the cell-free nucleic acids in the plurality of control samples; (b) setting a plurality of candidate length ranges, and determining a percentage of the cell-free nucleic acids, obtained from each of the plurality of control samples, present in each candidate length range; (c) determining a correlation coefficient between each candidate length range and the fraction of the cell-free nucleic acids from the predetermined source, based on the percentage of the cell-free nucleic acids, obtained from each of the plurality of control samples, present in each candidate length range and the fraction of the cell-free nucleic acids from the predetermined source in the control samples; and (d) determining a candidate length range with the largest correlation coefficient as the predetermined range. Therefore, the predetermined range can be determined accurately and efficiently.

In embodiments of the present disclosure, the candidate length range is of a span of 5 bp to 20 bp.

In embodiments of the present disclosure, determining the fraction of the cell-free nucleic acids from the predetermined source in the biological sample based on the number of the cell-free nucleic acids in the length falling into the predetermined range further includes:

determining a percentage of the cell-free nucleic acids present in the predetermined range based on the number of cell-free nucleic acids in the length falling into the predetermined range; and

determining the fraction of the cell-free nucleic acids from the predetermined source in the biological sample, based on the percentage of the cell-free nucleic acids present in the predetermined range, according to a predetermined function, wherein the predetermined function is determined based on the plurality of control samples. Therefore, the fraction of the cell-free nucleic acids from the predetermined source in the biological sample can be determined efficiently, which gives rise to an accurate and reliable result and good reproducibility.

In embodiments of the present disclosure, the predetermined function is obtained by following steps:

(i) determining the percentage of the cell-free nucleic acids, obtained from each control sample, present in the predetermined range; and

(ii) fitting the percentage of the cell-free nucleic acids, obtained from each control sample, present in the predetermined range with the known fraction of the cell-free nucleic acid from the predetermined source, to determine the predetermined function. Therefore, the predetermined function can be determined accurately and efficiently, which is conducive to subsequent steps.

In embodiments of the present disclosure, the percentage of the cell-free nucleic acids, obtained from each control sample, present in the predetermined range is fitted with the known fraction of the cell-free nucleic acid from the predetermined source by a linear fitting.

In embodiments of the present disclosure, the cell-free nucleic acid from the predetermined source is cell-free fetal nucleic acid obtained from a peripheral blood sample of a pregnant woman, and the predetermined range is 185 bp to 204 bp. Therefore, the fraction of the cell-free nucleic acids from the predetermined source in the biological sample can be determined accurately based on the predetermined range. In embodiments of the present disclosure, the predetermined function is d=0.0334*p+1.6657, where d represents a fraction of cell-free fetal nucleic acids, and p represents a percentage of cell-free nucleic acid present in the predetermined range. The fraction of the cell-free nucleic acids from the predetermined source in the biological sample can be efficiently determined based on the predetermined function, which gives rise to an accurate and reliable result and good reproducibility.

In embodiments of the present disclosure, the control sample is a peripheral blood sample obtained from a pregnant woman in which the fraction of the cell-free fetal nucleic acids is known. Therefore, the predetermined range is determined accurately.

In embodiments of the present disclosure, the control sample is a peripheral blood sample obtained from a pregnant woman with a normal male fetus, in which the fraction of the cell-free fetal nucleic acids is known to be determined by chromosome Y. Therefore, the predetermined range is determined accurately.

In embodiments of the present disclosure, the fraction of cell-free nucleic acids in the control sample is a cell-free fetal DNA fraction which is estimated by chromosome Y. Therefore, the predetermined range can be determined by efficiently utilizing the fraction of cell-free nucleic acids of the control sample, and then the number of the cell-free nucleic acids in the length falling into the predetermined range and the cell-free fetal DNA fraction in a simple obtained from a pregnant woman under detection can be further determined.

In a second aspect, the present disclosure further provides a device for determining a fraction of cell-free nucleic acids from a predetermined source in a biological sample. In embodiments of the present disclosure, the device includes: a sequencing apparatus, configured to sequence cell-free nucleic acids contained in the biological sample, so as to obtain a sequencing result consisting of a plurality of sequencing data; a counting apparatus, connected to the sequencing apparatus and configured to determine the number of the cell-free nucleic acids in a length falling into a predetermined range in the biological sample based on the sequencing result; and an apparatus for determining a fraction of cell-free nucleic acids, connected to the counting apparatus and configured to determine the fraction of the cell-free nucleic acids from the predetermined source in the biological sample based on the number of the cell-free nucleic acids in the length falling into the predetermined range. The inventors have surprisingly found that, the device of the present disclosure is suitable to carry out the method for determining a fraction of cell-free nucleic acids from a predetermined source in a biological sample described hereinbefore, by which the fraction of the cell-free nucleic acids from the predetermined source in the biological sample, especially the fraction of the cell-free fetal nucleic acids in a peripheral blood sample obtained from a pregnant woman, or the fraction of cell-free tumor derived nucleic acids in a peripheral blood sample obtained from a subject suffering from tumor, suspected to suffer from tumor or subjected to tumor screening can be accurately and efficiently determined.

In embodiments of the present disclosure, the biological sample is a peripheral blood sample.

In embodiments of the present disclosure, the cell-free nucleic acid from the predetermined source is selected from one of the followings: cell-free fetal nucleic acids or cell-free maternal nucleic acids in a peripheral blood sample obtained from a pregnant woman, or cell-free tumor derived nucleic acids or cell-free non-tumor derived nucleic acids in a peripheral blood sample obtained from a subject suffering from tumor, suspected to suffer from tumor or subjected to tumor screening. Therefore, the fraction of the cell-free fetal nucleic acids in a peripheral blood sample obtained from a pregnant woman, or the fraction of cell-free tumor derived nucleic acids in a peripheral blood sample obtained from a subject suffering from tumor, suspected to suffer from tumor or subjected to tumor screening can be easily determined.

In embodiments of the present disclosure, the nucleic acids are DNA.

In embodiments of the present disclosure, the sequencing result includes lengthen of the cell-free nucleic acids.

In embodiments of the present disclosure, the cell-free nucleic acids in the biological sample are sequenced by paired-end sequencing, single-end sequencing or single molecule sequencing. Therefore, lengthen of the cell-free nucleic acids may be obtained easily, which is conducive to subsequent steps.

In embodiments of the present disclosure, the counting apparatus further includes: an aligning unit, configured to align the sequencing result to a reference genome, so as to construct a dataset consisting of a plurality of uniquelymapped reads, where each read in the dataset can be mapped to a position of the reference genome only; a first length determining unit, connected to the aligning unit and configured to determine a length of the cell-free nucleic acid corresponding to each uniquelymapped read in the dataset; and a number determining unit, connected to the first length determining unit and configured to determine the number of the cell-free nucleic acids in the length falling into the predetermined range. Therefore, the number of the cell-free nucleic acids in the length falling into the predetermined range in the biological sample can be determined easily, which gives rise to an accurate and reliable result and good reproducibility.

In embodiments of the present disclosure, the first length determining apparatus is configured to determine the length of each read uniquely mapped to the reference genome as the length of the cell-free nucleic acid corresponding to the read. Therefore, the length of the cell-free nucleic acid corresponding to each uniquely-mapped read in the dataset can be determined accurately.

In embodiments of the present disclosure, in the case that the cell-free nucleic acids in the biological sample are sequenced by the paired-end sequencing, the first length determining unit further includes: a 5′-end position determining module, configured to determine a position, corresponding to the reference genome, of 5′-end of the cell-free nucleic acid, based on sequencing data at one end of each uniquelymapped read obtained in the paired-end sequencing; a 3′-end position determining module, connected to the 5′-end position determining module and configured to determine a position, corresponding to the reference genome, of 3′-end of the cell-free nucleic acid, based on sequencing data at the other end of same uniquely mapped read obtained in the paired-end sequencing; and a length calculating module, connected to the 3′-end position determining module and configured to determine the length of the cell-free nucleic acid based on the position of 5′-end of the cell-free nucleic acid and the position of 3′-end of the cell-free nucleic acid. Therefore, the length of the cell-free nucleic acid corresponding to each uniquely mapped read in the dataset can be determined accurately.

In embodiments of the present disclosure, the device further includes a predetermined range determining apparatus configured to determine the predetermined range based on a plurality of control samples, in each of which the fraction of the cell-free nucleic acids from the predetermined source is known, optionally, the predetermined range is determined based on at least 20 control samples.

In embodiments of the present disclosure, the predetermined range determining apparatus further includes: a second length determining unit, configured to determine lengths of the cell-free nucleic acids in the plurality of control samples; a first percentage determining unit, connected to the second length determining unit and configured to set a plurality of candidate length ranges and determine a percentage of the cell-free nucleic acids, obtained from each of the plurality of control samples, present in each candidate length range; a correlation coefficient determining unit, connected to the first percentage determining unit and configured to determine a correlation coefficient between each candidate length range and the fraction of the cell-free nucleic acids from the predetermined source, based on the percentage of the cell-free nucleic acids, obtained from each of the plurality of control samples, present in each candidate length range and the fraction of the cell-free nucleic acids from the predetermined source in the control samples; and a predetermined range determining unit, connected to the correlation coefficient determining unit and configured to select a candidate length range with the largest correlation coefficient as the predetermined range. Therefore, the predetermined range can be determined accurately and efficiently.

In embodiments of the present disclosure, the candidate length range is of a span of 1 bp to 20 bp.

In embodiments of the present disclosure, the plurality of candidate length ranges is of a step size of 1 bp to 2 bp.

In embodiments of the present disclosure, the apparatus for determining a fraction of cell-free nucleic acids further includes: a second percentage determining unit, configured to determine a percentage of the cell-free nucleic acids present in the predetermined range based on the number of cell-free nucleic acids in the length falling into the predetermined range; and a unit for calculating a fraction of cell-free nucleic acids, connected to the second percentage determining unit and configured to determine the fraction of the cell-free nucleic acids from the predetermined source in the biological sample, based on the percentage of the cell-free nucleic acids present in the predetermined range, according to a predetermined function, in which the predetermined function is determined based on the plurality of control samples. Therefore, the fraction of the cell-free nucleic acids from the predetermined source in the biological sample can be determined efficiently, which gives rise to an accurate and reliable result and good reproducibility.

In embodiments of the present disclosure, the device further includes a predetermined function determining apparatus, which includes: a third percentage determining unit, configured to determine the percentage of the cell-free nucleic acids, obtained from each control sample, present in the predetermined range; and a fitting unit, connected to the third percentage determining unit and configured to fit the percentage of the cell-free nucleic acids, obtained from each control sample, present in the predetermined rangewith the known fraction of the cell-free nucleic acid from the predetermined source, to determine the predetermined function. Therefore, the predetermined function can be determined accurately and efficiently, which is conducive to subsequent steps.

In embodiments of the present disclosure, the percentage of the cell-free nucleic acids, obtained from each control sample, present in the predetermined range is fitted with the known fraction of the cell-free nucleic acid from the predetermined source by a linear fitting.

In embodiments of the present disclosure, the cell-free nucleic acid from the predetermined source is cell-free fetal nucleic acid obtained from a peripheral blood sample of a pregnant woman, and the predetermined range is 185 bp to 204 bp. Therefore, the fraction of the cell-free nucleic acids from the predetermined source in the biological sample can be determined accurately based on the predetermined range.

In embodiments of the present disclosure, the predetermined function is d=0.0334*p+1.6657, where d represents a fraction of cell-free fetal nucleic acids, and p represents a percentage of cell-free nucleic acid present in the predetermined range. The fraction of the cell-free nucleic acids from the predetermined source in the biological sample can be efficiently determined based on the predetermined function, which gives rise to an accurate and reliable result and good reproducibility.

In embodiments of the present disclosure, the control sample is a peripheral blood sample obtained from a pregnant woman in which the fraction of the cell-free fetal nucleic acids is known.

In embodiments of the present disclosure, the control sample is a peripheral blood sample obtained from a pregnant woman with a normal male fetus, in which the fraction of the cell-free fetal nucleic acids is known to be determined by chromosome Y. Therefore, the predetermined range is determined accurately.

In embodiments of the present disclosure, the fraction of cell-free nucleic acids in the control sample is a cell-free fetal DNA fraction which is determined by a device suitable for estimation with chromosome Y. Therefore, the predetermined range can be determined by efficiently utilizing the fraction of cell-free nucleic acids of the control sample, and then the number of the cell-free nucleic acids in the length falling into the predetermined range and the cell-free fetal DNA fraction in a simple obtained from a pregnant woman under detection can be further determined.

It should be noted that, the method and device for determining the fraction of cell-free nucleic acids in a biological sample according to the present disclosure at least have the following advantages.

1) Universality: cell-free fetal (in particular female fetus) DNA fractions in all samples meeting the quality control can be estimated.

2) Accuracy of NIPT detection can be improved.

3) Operational simplicity: the cell-free fetal DNA fractions can be estimated directly merely using offline data.

In a third aspect, the present disclosure provides a method for determining sexuality of twins. In embodiments of the present disclosure, the method includes: performing sequencing on cell-free nucleic acids contained in a peripheral blood sample obtained from a pregnant woman with twins, so as to obtain a sequencing result consisting of a plurality of sequencing data; determining a first cell-free fetal DNA fraction based on the sequencing data, by the method hereinbefore for determining the fraction of cell-free nucleic acids in a biological sample; determining a second cell-free fetal DNA fraction based on a sequencing data derived from chromosome Y in the sequencing result; and determining the sexuality of the twins based on the first cell-free fetal DNA fraction and the second cell-free fetal DNA fraction. The inventors have surprisingly found that, sexuality of twins in a pregnant woman can be accurately and efficiently determined by the method of the present disclosure.

In embodiments of the present disclosure, the second cell-free fetal DNA fraction is determined according to the following formula:

fra.chry=(chry.ER %−Female.chry.ER %)/(Man.chry.ER %−Female.chry.ER %)*100%,

where fra.chry represents the second cell-free fetal DNA fraction, chry.ER % represents a percentage of the sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man to total sequencing data thereof.

Therefore, the second cell-free fetal DNA fraction can be determined accurately.

In embodiments of the present disclosure, determining the sexuality of the twins based on the first cell-free fetal DNA fraction and the second cell-free fetal DNA fraction further includes: (a) determining a ratio of the second cell-free fetal DNA fraction to the first cell-free fetal DNA fraction; and (b) determining the sexuality of the twins by comparing the ratio determined in (a) with a first threshold and a second threshold predetermined. Therefore, the sexuality of the twins can be determined efficiently.

In embodiments of the present disclosure, the first threshold is determined based on a pluratity of control samples obtained from pregnant women known with female twins, and the second threshold is determined based on a pluratity of control samples obtained from pregnant women known with male twins.

In embodiments of the present disclosure, both fetuses of the twins are female if the ratio of the second cell-free fetal DNA fraction to the first cell-free fetal DNA fraction is lower than the first threshold, both fetuses of the twins are male if the ratio of the second cell-free fetal DNA fraction to the first cell-free fetal DNA fraction is greater than the second threshold, and the twins include a male fetus and a female fetus if the ratio of the second cell-free fetal DNA fraction to the first cell-free fetal DNA fraction is equal to the first threshold or the second threshold, or between the first threshold and the second threshold.

In embodiments of the present disclosure, the first threshold is 0.35, and the second threshold is 0.7.

In a fourth aspect, the present disclosure provides a system for determining sexuality of twins. In embodiments of the present disclosure, the system includes:

a first cell-free fetal DNA fraction determining device, being the device hereinbefore for determining the fraction of cell-free nucleic acids in the biological sample, and configured to sequence cell-free nucleic acids contained in a peripheral blood sample obtained from a pregnant woman with twins, so as to obtain a sequencing result consisting of a plurality of sequencing data, and configured to determine a first cell-free fetal DNA fraction based on the sequencing data;

a second cell-free fetal DNA fraction determining device, configured to determine a second cell-free fetal DNA fraction based on a sequencing data derived from chromosome Y in the sequencing result; and

a sexuality determining device, configured to determine the sexuality of the twins based on the first cell-free fetal DNA fraction and the second cell-free fetal DNA fraction.

The inventors have surprisingly found that, sexuality of twins in a pregnant woman can be accurately and efficiently determined by the system of the present disclosure.

In embodiments of the present disclosure, the second cell-free fetal DNA fraction is determined according to the following formula:

fra.chry=(chry.ER %—Female.chry.ER %)/(Man.chry.ER %−Female.chry.ER %)*100%,

where fra.chry represents the second cell-free fetal DNA fraction, chry.ER % represents a percentage of the sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man to total sequencing data thereof. Therefore, the second cell-free fetal DNA fraction can be determined accurately.

In embodiments of the present disclosure, the sexuality determining device further includes: a ratio determining unit, configured to determine a ratio of the second cell-free fetal DNA fraction to the first cell-free fetal DNA fraction; and a comparison unit, configured to compare the ratio determined by the ratio determining unit with a first threshold and a second threshold predetermined, so as to determine the sexuality of the twins. Therefore, the sexuality of the twins can be determined efficiently.

In embodiments of the present disclosure, the first threshold is determined based on a pluratity of control samples obtained from pregnant women known with female twins, and the second threshold is determined based on a pluratity of control samples obtained from pregnant women known with male twins.

In embodiments of the present disclosure, both fetuses of the twins are female if the ratio of the second cell-free fetal DNA fraction to the first cell-free fetal DNA fraction is lower than the first threshold, both fetuses of the twins are male if the ratio of the second cell-free fetal DNA fraction to the first cell-free fetal DNA fraction is greater than the second threshold, and the twins include a male fetus and a female fetus if the ratio of the second cell-free fetal DNA fraction to the first cell-free fetal DNA fraction is equal to the first threshold or the second threshold, or between the first threshold and the second threshold.

In embodiments of the present disclosure, the first threshold is 0.35, and the second threshold is 0.7.

In a fifth aspect, the present disclosure provides a method for detecting a chromosome aneuploidy of twins. In embodiments of the present disclosure, the method includes:

performing sequencing on cell-free nucleic acids contained in a peripheral blood sample obtained from a pregnant woman with twins, so as to obtain a sequencing result consisting of a plurality of sequencing data;

determining a first cell-free fetal DNA fraction, based on the sequencing data, by the method hereinbefore for determining the fraction of cell-free nucleic acids in a biological sample;

determining a third cell-free fetal DNA fraction, based on a sequencing data derived from a predetermined chromosome in the sequencing result; and

determining whether the twins under detection have aneuploidy with respect to the predetermined chromosome based on the first cell-free fetal DNA fraction and the third cell-free fetal DNA fraction.

Therefore, the chromosome aneuploidy of twins can be detected acurately and efficiently.

In embodiments of the present disclosure, the third cell-free fetal DNA fraction is determined according to the following formula:

fra.chri=2*(chri.ER %/adjust.chri.ER %−1)*100%,

Where fra.chri represents the third cell-free fetal DNA fraction, i represents a serial number of the predetermined chromosome, and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with normal twins to total sequencing data thereof.

Therefore, the third cell-free fetal DNA fraction can be determined accurately.

In embodiments of the present disclosure, determining whether the twins under detection have aneuploidy with respect to the predetermined chromosome based on the first cell-free fetal DNA fraction and the third cell-free fetal DNA fraction further includes: (a) determining a ratio of the third cell-free fetal DNA fraction to the first cell-free fetal DNA fraction; and (b) determining whether the twins under detection have aneuploidy with respect to the predetermined chromosome by comparing the ratio determined in (a) with a third threshold and a fourth threshold predetermined. Therefore, the chromosome aneuploidy of twins can be detected efficiently.

In embodiments of the present disclosure, the third threshold is determined based on a pluratity of control samples obtained from pregnant women with twins known not to have aneuploidy with respect to the predetermined chromosome, and the fourth threshold is determined based on a pluratity of control samples obtained from pregnant women with twins known to have aneuploidy with respect to the predetermined chromosome.

In embodiments of the present disclosure, both fetuses of the twins have no aneuploidy with respect to the predetermined chromosome if the ratio of the third cell-free fetal DNA fraction to the first cell-free fetal DNA fraction is lower than the third threshold, both fetuses of the twins have aneuploidy with respect to the predetermined chromosome if the ratio of the third cell-free fetal DNA fraction to the first cell-free fetal DNA fraction is greater than the fourth threshold, and one fetus of the twins has the aneuploidy with respect to the predetermined chromosome, while the other fetus of the twins has no aneuploidy with respect to the predetermined chromosome if the ratio of the third cell-free fetal DNA fraction to the first cell-free fetal DNA fraction is equal to the third threshold or the fourth threshold, or between the third threshold and the fourth threshold.

In embodiments of the present disclosure, the third threshold is 0.35, and the fourth threshold is 0.7.

In embodiments of the present disclosure, the predetermined chromosome is at least one selected from chromosomes 18, 21 and 23.

In a sixth aspect, the present disclosure provides a system for determining a chromosome aneuploidy of twins. In embodiments of the present disclosure, the system includes:

a first cell-free fetal DNA fraction determining device, being the device hereinbefore for determining the fraction of cell-free nucleic acids in the biological sample, and configured to sequence cell-free nucleic acids contained in a peripheral blood sample obtained from a pregnant woman with twins, so as to obtain a sequencing result consisting of a plurality of sequencing data, and configured to determine a first cell-free fetal DNA fraction based on the sequencing data;

a third cell-free fetal DNA fraction determining device, configured to determine a third cell-free fetal DNA fraction based on a sequencing data derived from a predetermined chromosome in the sequencing result; and

a first aneuploidy determining device, configured to determine whether the twins under detection have aneuploidy with respect to the predetermined chromosome based on the first cell-free fetal DNA fraction and the third cell-free fetal DNA fraction. The inventors have surprisingly found that, the chromosome aneuploidy of twins can be detected ccurately and efficiently by the system according to the present disclosure.

In embodiments of the present disclosure, the third cell-free fetal DNA fraction is determined according to the following formula:

fra.chri=2*(chri.ER %/adjust.chri.ER %−1)*100%,

Where fra.chri represents the third cell-free fetal DNA fraction, i represents a serial number of the predetermined chromosome, and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with normal twins to total sequencing data thereof. Therefore, the third cell-free fetal DNA fraction can be determined accurately.

In embodiments of the present disclosure, the first aneuploidy determining device further includes:

a ratio determining unit, configured to determine a ratio of the third cell-free fetal DNA fraction to the first cell-free fetal DNA fraction; and

a comparison unit, configured to compare the ratio determined by the ratio determining unit with a third threshold and a fourth threshold predetermined, so as to determine whether the twins under detection have aneuploidy with respect to the predetermined chromosome. Therefore, the chromosome aneuploidy of twins can be detected efficiently.

In embodiments of the present disclosure, the third threshold is determined based on a pluratity of control samples obtained from pregnant women with twins known not to have aneuploidy with respect to the predetermined chromosome, and the fourth threshold is determined based on a pluratity of control samples obtained from pregnant women with twins known to have aneuploidy with respect to the predetermined chromosome.

In embodiments of the present disclosure, both fetuses of the twins have no aneuploidy with respect to the predetermined chromosome if the ratio of the third cell-free fetal DNA fraction to the first cell-free fetal DNA fraction is lower than the third threshold, both fetuses of the twins have aneuploidy with respect to the predetermined chromosome if the ratio of the third cell-free fetal DNA fraction to the first cell-free fetal DNA fraction is greater than the fourth threshold, and one fetus of the twins has the aneuploidy with respect to the predetermined chromosome, while the other fetus of the twins has no aneuploidy with respect to the predetermined chromosome if the ratio of the third cell-free fetal DNA fraction to the first cell-free fetal DNA fraction is equal to the third threshold or the fourth threshold, or between the third threshold and the fourth threshold. Therefore, the chromosome aneuploidy of twins can be detected efficiently.

In embodiments of the present disclosure, the third threshold is 0.35, and the fourth threshold is 0.7.

In embodiments of the present disclosure, the predetermined chromosome is at least one selected from chromosomes 18, 21 and 23.

In a seventh aspect, the present disclosure provides a method for determining a chromosome aneuploidy of twins. In embodiments of the present disclosure, the method includes:

performing sequencing on cell-free nucleic acids contained in a peripheral blood sample obtained from a pregnant woman with twins, so as to obtain a sequencing result consisting of a plurality of sequencing data;

determining a fraction xᵢ, of the number of sequencing data derived from chromosome i in the sequencing result to total sequencing data, where i represents a serial number of the chromosome, and i is any integer in the range of 1 to 22;

determining a T score of the chromosome i according to Tᵢ=(xᵢ−μᵢ)/σᵢ, where i represents the serial number of the chromosome and i is any integer in the range of 1 to 22, μᵢ represents an average percentage of sequencing data of the chromosome i selected as a reference system in a reference database to total sequencing data thereof, σᵢ represents a standard deviation of percentages of the sequencing data of the chromosome i selected as the reference system in the reference database to total sequencing data thereof,

determining an L score of the chromosome i according to Lᵢ=log(d(Tᵢ, a))/log(d (T2ᵢ, a)), where i represents the serial number of the chromosome and i is any integer in the range of 1 to 22, T2ᵢ=(xᵢ−μᵢ(1+fra/2))/σᵢ; d(Tᵢ, a) and d(T2ᵢ, a) represent t distribution probability density function, where a represents degree of freedom, fra represents a first cell-free fetal DNA fraction determined by the method hereinbefore for determining the fraction of cell-free nucleic acids in a biological sample or a fetal fraction estimated by chromosome Y (fra.chrY %), fra.chry=(chry.ER %−Female.chr.ER %)/(Man.chry.UR %−Female.ch.ER %)*100%, where fra.chry represents a cell-free fetal DNA fraction, ch.ER % represents a percentage of sequencing data derived from chromosome Y in the sequencing result to said total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;

plotting a four-quadrant diagram with T as vertical coordinate and L as horizontal coordinate by zoning with a first straight line where T=predetermined fifth threshold and a second straight line where L=predetermined sixth threshold, wherein both fetuses of the twins are determined to have trisome if a sample under detection is determined to be of the T score and the L score falling into a first quadrant; one fetus of the twins is determined to be of trisome and the other fetus of the twins is determined to be normal if a sample under detection is determined to be of the T score and the L score falling into a second quadrant; both fetuses of the twins are determined to be normal if a sample under detection is determined to be of the T score and the L score falling into a third quadrant; the twins are determined to have a low fetal fraction if a sample under detection is determined to be of the T score and the L score falling into a fourth quadrant, such a result is not adopted. The inventors have surprisingly found that, the detection of the chromosome aneuploidy of twins of a pregnant woman and the determination of whether the twins under detection have aneuploidy with respect to the predetermined chromosome can be achieved accurately and efficiently by the method for determining the chromosome aneuploidy of twins according to the present disclosure.

In an eighth aspect, the present disclosure provides a system for determining a chromosome aneuploidy of twins. In embodiments of the present disclosure, the system includes:

an xᵢ value determining device, configured to sequence cell-free nucleic acids contained in a peripheral blood sample obtained from a pregnant woman with twins, so as to obtain a sequencing result consisting of a plurality of sequencing data, and configured to determine a fraction xᵢ of the number of sequencing data derived from chromosome i in the sequencing result to total sequencing data, where i represents a serial number of the chromosome and i is any integer in the range of 1 to 22;

a T score determining device, configured to determine a T score of the chromosome i according to Tᵢ=(xᵢ−μᵢ)/σᵢ, where i represents the serial number of the chromosome and i is any integer in the range of 1 to 22, μᵢ represents an average percentage of sequencing data of the chromosome i selected as a reference system in a reference database to total sequencing data thereof, σᵢ represents a standard deviation of percentages of the sequencing data of the chromosome i selected as the reference system in the reference database to total sequencing data thereof,

an L score determining device, configured to determine an L score of the chromosome i according to Lᵢ=log(d(Tᵢ, a))/log(d(T2ᵢ,a)), where i represents the serial number of the chromosome and i is any integer in the range of 1 to 22, T2ᵢ=(xᵢ−μᵢ*(1+fra/2))/σᵢ; d(Tᵢ, a) and d(T2ᵢ, a) represent t distribution probability density function, where a represents degree of freedom, fra represents a cell-free fetal DNA fraction determined by the method hereinbefore or a fetal fraction estimated by chromosome Y (fra.chrY %),

fra.chry=(chry.ER %−Female.chr.ER %)/(Man.chry.UR %−Female.chry.ER %)*100%,

where fra.chry represents a cell-free fetal DNA fraction, chry.ER % represents a percentage of sequencing data derived from chromosome Y in the sequencing result to said total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;

a second aneuploidy determining device, configured to plot a four-quadrant diagram with T as vertical coordinate and L as horizontal coordinate by zoning with a first straight line where T=predetermined fifth threshold and a second straight line where L=predetermined sixth threshold,

wherein both fetuses of the twins are determined to have trisome if a sample under detection is determined to be of the T score and the L score falling into a first quadrant; one fetus of the twins is determined to have trisome and the other fetus of the twins is determined to be normal if a sample under detection is determined to be of the T score and the L score falling into a second quadrant; both fetuses of the twins are determined to be normal if a sample under detection is determined to be of the T score and the L score falling into a third quadrant; the twins are determined to have a low fetal fraction if a sample under detection is determined to be of the T score and the L score falling into a fourth quadrant, such a result is not adopted. The inventors have surprisingly found that, the detection of the chromosome aneuploidy of twins of a pregnant woman and the determination of whether the twins under detection have aneuploidy with respect to the predetermined chromosome can be achieved accurately and efficiently by the system for determining the chromosome aneuploidy of twins according to the present disclosure.

In a ninth aspect, the present disclosure provides a method for detecting fetal chimera. In embodiments of the present disclosure, the method includes:

performing sequencing on cell-free nucleic acids contained in a peripheral blood sample obtained from a pregnant woman with a fetus, optionally a male fetus, so as to obtain a sequencing result consisting of a plurality of sequencing data;

determining a first cell-free fetal DNA fraction, based on the sequencing data, by the method hereinbefore, or estimating a fetal fraction by chromosome Y (fra.chrY %) as the first cell-free fetal DNA fraction according to the following formula:

fra.chry=(chry.ER %−Female.chr.ER %)/(Man.chry.UR %−Female.chry.ER %)*100%,

where fra.chry represents the first cell-free fetal DNA fraction, chry.ER % represents a percentage of sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;

determining a third cell-free fetal DNA fraction based on sequencing data derived from a predetermined chromosome in the sequencing result; and

determining whether the fetus under detection has fetal chimera with respect to the predetermined chromosome based on the first cell-free fetal DNA fraction and the third cell-free fetal DNA fraction.

Therefore, whether the fetus under detection has fetal chimera with respect to a specific chromosome can be analyzed accurately.

In embodiments of the present disclosure, the method may further have the following additional technical features:

In embodiments of the present disclosure, the third cell-free fetal DNA fraction is determined by the following formula:

fra.chri=2*(chri.ER %/adjust.chri.ER %−1)*100%,

where fra.chri represents the third cell-free fetal DNA fraction, i represents a serial number of the predetermined chromosome and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with a normal fetus to total sequencing data thereof. Therefore, whether the fetus under detection has fetal chimera with respect to the specific chromosome can be analyzed with further improved efficiency.

In embodiments of the present disclosure, determining whether the fetus under detection has fetal chimera with respect to the predetermined chromosome based on the first cell-free fetal DNA fraction and the third cell-free fetal DNA fraction further includes: (a) determining a ratio of the third cell-free fetal DNA fraction to the first cell-free fetal DNA fraction; and (b) determining whether the fetus under detection has chimera with respect to the predetermined chromosome by comparing the ratio determined in (a) with a plurality of predetermined thresholds. Therefore, whether the fetus under detection has fetal chimera with respect to the specific chromosome can be analyzed with further improved efficiency.

In embodiments of the present disclosure, the plurality of predetermined thresholds includes at least one selected from:

a seventh threshold, determined based on a pluratity of control samples with the predetermined chromosome known to be of complete monosome,

an eighth threshold, determined based on a pluratity of control samples with the predetermined chromosome known to be of monosome chimera,

a ninth threshold, determined based on a pluratity of control samples with the predetermined chromosome known to be normal,

a tenth threshold, determined based on a pluratity of control samples with the predetermined chromosome known to be of complete trisome.

In embodiments of the present disclosure, the predetermined chromosome of the fetus under detection is of complete monosome, if the ratio of the third cell-free fetal DNA fraction to the first cell-free fetal DNA fraction is lower than the seventh threshold;

the predetermined chromosome of the fetus under detection is of monosome chimera, if the ratio of the third cell-free fetal DNA fraction to the first cell-free fetal DNA fraction is not lower than the seventh threshold and not greater than the eighth threshold;

the predetermined chromosome of the fetus under detection is normal, if the ratio of the third cell-free fetal DNA fraction to the first cell-free fetal DNA fraction is greater than the eighth threshold and lower than the ninth threshold;

the predetermined chromosome of the fetus under detection is of trisome chimera, if the ratio of the third cell-free fetal DNA fraction to the first cell-free fetal DNA fraction is not lower than the ninth threshold and not greater than the tenth threshold; and

the predetermined chromosome of the fetus under detection is of complete trisome, if the ratio of the third cell-free fetal DNA fraction to the first cell-free fetal DNA fraction is greater than the tenth threshold.

In embodiments of the present disclosure, the seventh threshold at least is −1 and lower than 0, optionally is −0.85;

the eighth threshold is greater than the seventh threshold and lower than 0, optionally is −0.3;

the ninth threshold is greater than 0 and lower than 1, optionally is 0.3;

the tenth threshold is greater than the ninth threshold and lower than 1, optionally is 0.85. Therefore, whether the fetus under detection has fetal chimera with respect to a specific chromosome can be analyzed with further improved efficiency.

In a tenth aspect, the present disclosure provides a system for detecting fetal chimer. In embodiments of the present disclosure, the system includes:

a first cell-free fetal DNA fraction determining device, being the device hereinbefore for determining the fraction of cell-free nucleic acids in the biological sample, and configured to sequence cell-free nucleic acids contained in a peripheral blood sample obtained from a pregnant woman with fetus, so as to obtain a sequencing result consisting of a plurality of sequencing data, and configured to determine a first cell-free fetal DNA fraction based on the sequencing data, or configured to estimate a fetal fraction by chromosome Y (fra.chrY %) as the first cell-free fetal DNA fraction according to the following formula:

fra.chry=(chry.ER %−Female.chr.ER %)/(Man.chry.UR %−Female.ch.ER %)*100%,

where fra.chry represents the first cell-free fetal DNA fraction, chry.ER % represents a percentage of sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;

a third cell-free fetal DNA fraction determining device, configured to determine a third cell-free fetal DNA fraction based on sequencing data derived from a predetermined chromosome in the sequencing result; and

a chimera determining device, configured to determine whether the fetus under detection has fetal chimera with respect to the predetermined chromosome based on the first cell-free fetal DNA fraction and the third cell-free fetal DNA fraction.

According to embodiments of the present disclosure, the method hereinbefore for determining fetal chimera can be efficiently carried out by the system above, such that whether the fetus under detection has fetal chimera can be efficiently analyzed.

In embodiments of the present disclosure, the system above for detecting fetal chimera may further include the following additional technical features.

In embodiments of the present disclosure, the third cell-free fetal DNA fraction is determined by the following formula:

fra.chri=2*(chri.ER %/adjust.chri.ER %−1)*100%,

where fra.chri represents the third cell-free fetal DNA fraction, i represents a serial number of the predetermined chromosome and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with a normal fetus to total sequencing data thereof. Therefore, whether the fetus under detection has fetal chimera with respect to a specific chromosome can be analyzed with further improved efficiency.

In embodiments of the present disclosure, the chimera determining device includes:

a ratio determining unit, configured to determine a ratio of the third cell-free fetal DNA fraction to the first cell-free fetal DNA fraction; and

a comparison unit, configured to compare the ratio determined by the ratio determining unit with a plurality of predetermined thresholds, so as to determine whether the fetus under detection has chimera with respect to the predetermined chromosome.

In embodiments of the present disclosure, the plurality of predetermined thresholds includes at least one selected from:

a seventh threshold, determined based on a pluratity of control samples with the predetermined chromosome known to be of complete monosome,

an eighth threshold, determined based on a pluratity of control samples with the predetermined chromosome known to be of monosome chimera,

a ninth threshold, determined based on a pluratity of control samples with the predetermined chromosome known to be normal,

a tenth threshold, determined based on a pluratity of control samples with the predetermined chromosome known to be of complete trisome,

In embodiments of the present disclosure, the predetermined chromosome of the fetus under detection is of complete monosome, if the ratio of the third cell-free fetal DNA fraction to the first cell-free fetal DNA fraction is lower than the seventh threshold;

the predetermined chromosome of the fetus under detection is of monosome chimera, if the ratio of the third cell-free fetal DNA fraction to the first cell-free fetal DNA fraction is not lower than the seventh threshold and not greater than the eighth threshold;

the predetermined chromosome of the fetus under detection is normal, if the ratio of the third cell-free fetal DNA fraction to the first cell-free fetal DNA fraction is greater than the eighth threshold and lower than the ninth threshold;

the predetermined chromosome of the fetus under detection is of trisome chimera, if the ratio of the third cell-free fetal DNA fraction to the first cell-free fetal DNA fraction is not lower than the ninth threshold and not greater than the tenth threshold; and

the predetermined chromosome of the fetus under detection is of complete trisome, if the ratio of the third cell-free fetal DNA fraction to the first cell-free fetal DNA fraction is greater than the tenth threshold.

In embodiments of the present disclosure, the seventh threshold is greater than −1 and lower than 0, optionally is −0.85;

the eighth threshold is greater than the seventh threshold and lower than 0, optionally is −0.3;

the ninth threshold is greater than 0 and lower than 1, optionally is 0.3;

the tenth threshold is greater than the ninth threshold and lower than 1, optionally is 0.85.

Therefore, whether the fetus under detection has fetal chimera with respect to a specific chromosome can be analyzed with further improved efficiency.

In an eleventh aspect, the present disclosure provides a method for detecting fetal chimera. In embodiments of the present disclosure, the method includes:

performing sequencing on cell-free nucleic acids contained in a peripheral blood sample obtained from a pregnant woman with a fetus, so as to obtain a sequencing result consisting of a plurality of sequencing data;

determining a fraction xᵢ, of the number of sequencing data derived from chromosome i in the sequencing result to total sequencing data, where i represents a serial number of the chromosome, and i is any integer in the range of 1 to 22;

determining a T score of the chromosome i according to Tᵢ=(xᵢ−μᵢ)/σᵢ, where i represents the serial number of the chromosome and i is any integer in the range of 1 to 22, μᵢ represents an average value of percentages of sequencing data of the chromosome i selected as a reference system in a reference database to total sequencing data thereof, σᵢ represents a standard deviation of percentages of the sequencing data of the chromosome i selected as the reference system in the reference database to total sequencing data thereof;

determining an L score of the chromosome i according to Lᵢ=log(d(Tᵢ, a))/log(d(T2ᵢ,a)), where i represents the serial number of the chromosome and i is any integer in the range of 1 to 22, T2ᵢ=(xᵢ−μᵢ*(1+fra/2))/σᵢ; d(Tᵢ, a) and d(T2ᵢ, a) represent t distribution probability density function, a represents degree of freedom, fra represents a cell-free fetal DNA fraction determined by the method hereinbefore for determining the fraction of cell-free nucleic acids in the biological sample;

plotting a four-quadrant diagram with T as vertical coordinate and L as horizontal coordinate by zoning with a third straight line where T=predetermined eleventh threshold and a fourth straight line where L=predetermined twelfth threshold, when the T score is not greater than 0,

wherein the fetus is determined to have complete monosome or monosome chimera with respect to the predetermined chromosome, if a sample under detection is determined to be of the T score and the L score falling into a first quadrant;

the fetus is determined to have monosome chimera with respect to the predetermined chromosome, if a sample under detection is determined to be of the T score and the L score falling into a second quadrant;

the fetus is determined to be normal with respect to the predetermined chromosome, if a sample under detection is determined to be of the T score and the L score falling into a third quadrant;

the fetus is determined to have a low fetal fraction if a sample under detection is determined to be of the T score and the L score falling into a fourth quadrant, such a result is not adopted,

plotting a four-quadrant diagram with T as vertical coordinate and L as horizontal coordinate by zoning with a fifth straight line where T=predetermined thirteenth threshold and a sixth straight line where L=predetermined fourteenth threshold, when the T score is greater than 0,

wherein the fetus is determined to have complete trisome or trisome chimera with respect to the predetermined chromosome, if a sample under detection is determined to be of the T score and the L score falling into a first quadrant;

the fetus is determined to have trisome chimera with respect to the predetermined chromosome, if a sample under detection is determined to be of the T score and the L score falling into a second quadrant;

the fetus is determined to be normal with respect to the predetermined chromosome, if a sample under detection is determined to be of the T score and the L score falling into a third quadrant;

the fetus is determined to have a low fetal fraction if a sample under detection is determined to be of the T score and the L score falling into a fourth quadrant, such a result is not adopted,

optionally, the eleventh threshold and the thirteenth threshold each independently is 3, and the twelfth threshold and the fourteenth threshold each independently is 1.

Therefore, situations on the fetal chimera can be analyzed efficiently.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the drawings, in which:

FIG. 1 is a flow chart showing a method for determining a fraction of cell-free nucleic acids in a biological sample according to an embodiment of the present disclosure;

FIG. 2 is a flow chart showing a method for determining the number of cell-free nucleic acids in a length falling into a predetermined range according to an embodiment of the present disclosure;

FIG. 3 is a flow chart showing a method for determining the length of the cell-free nucleic acid according to an embodiment of the present disclosure;

FIG. 4 is a flow chart showing a method for determining a predetermined range according to an embodiment of the present disclosure;

FIG. 5 is a flow chart showing a method for determining a fraction of cell-free nucleic acids from a predetermined source in a biological sample according to an embodiment of the present disclosure;

FIG. 6 is a flow chart showing a method for determining a predetermined function according to an embodiment of the present disclosure;

FIG. 7 is a structural diagram of a device for determining a fraction of cell-free nucleic acids from a predetermined source in a biological sample according to an embodiment of the present disclosure;

FIG. 8 is a structural diagram of a counting apparatus according to an embodiment of the present disclosure;

FIG. 9 is a structural diagram of a first length determining unit according to an embodiment of the present disclosure;

FIG. 10 is a structural diagram of a predetermined range determining apparatus according to an embodiment of the present disclosure;

FIG. 11 is a structural diagram of an apparatus for determining a fraction of cell-free nucleic acids according to an embodiment of the present disclosure;

FIG. 12 is a structural diagram of a predetermined function determining apparatus according to an embodiment of the present disclosure;

FIG. 13 is a linear fitting diagram of correlation coefficient between the cell-free fetal DNA fraction estimated by chromosome Y and percentage of the percentage of DNA molecules present in 185 bp-204 bp for each sample obtained from 37 pregnant women known with a normal male fetus, according to an embodiment of the present disclosure; and

FIGS. 14-16 are four-quadrant diagrams on L scores and L scores of 11 samples under detection according to an embodiment of the present disclosure.

### DETAILED DESCRIPTION

Embodiments of the present disclosure will be described in detail below, which are explanatory, illustrative, and used to generally explain the present disclosure, thus shall not be construed to limit the present disclosure.

Method for Determining a Fraction of Cell-Free Nucleic Acids in a Biological Sample

According to embodiments of a first aspect of the present disclosure, a method for determining a fraction of cell-free nucleic acids from a predetermined source in a biological sample is provided. The inventors have surprisingly found that, the fraction of the cell-free nucleic acids from the predetermined source in the biological sample, especially the fraction of fetal nucleic acids in a peripheral blood sample obtained from a pregnant woman, or the fraction of tumor derived nucleic acids in a peripheral blood sample obtained from a subject suffering from tumor can be accurately and efficiently determined by the method of the present disclosure.

It should be noted that, expression “fraction of cell-free nucleic acids from the predetermined source in a biological sample” used herein refers to a fraction of the number of cell-free nucleic acids from specific source to the total number of cell-free nucleic acids in the biological sample. For example, if the biological sample is a peripheral blood obtained from a pregnant woman, the cell-free nucleic acids from the predetermined source are cell-free fetal nucleic acids, “fraction of cell-free nucleic acids from the predetermined source in a biological sample”, i.e. a fraction of cell-free fetal nucleic acids, means a fraction of the number of cell-free fetal nucleic acids to the total number of cell-free nucleic acids in the peripheral blood obtained from the pregnant woman, which sometimes also may be known as “cell-free fetal DNA fraction in the peripheral blood obtained from the pregnant woman” or cell-free fetal DNA fraction. As another example, if the biological sample is a peripheral blood sample obtained from a subject suffering from tumor, suspected to suffer from tumor or subjected to tumor screening, the cell-free nucleic acids from the predetermined source are cell-free tumor derived nucleic acids, “fraction of cell-free nucleic acids from the predetermined source in a biological sample”, i.e. a fraction of cell-free tumor derived nucleic acids, means a fraction of the number of cell-free tumor derived nucleic acids to the total number of cell-free nucleic acids in the peripheral blood sample obtained from the subject suffering from tumor, suspected to suffer from tumor or subjected to tumor screening. According to embodiments of the present disclosure and with reference to FIG. 1, the method includes the following steps.

S100: Nucleic Acid Sequencing

Cell-free nucleic acids in the biological sample are sequenced so as to obtain a sequencing result consisting of a plurality of sequencing data.

In embodiments of the present disclosure, the biological sample is a peripheral blood sample. In embodiments of the present disclosure, the cell-free nucleic acid from the predetermined source is cell-free fetal nucleic acids in a peripheral blood sample obtained from a pregnant woman or cell-free tumor derived nucleic acids. Therefore, the fraction of the cell-free fetal nucleic acids in the peripheral blood sample obtained from the pregnant woman, or the fraction of cell-free tumor derived nucleic acids in a peripheral blood sample obtained from a subject suffering from tumor, suspected to suffer from tumor or subjected to tumor screening can be easily determined. In some specific embodiments of the present disclosure, the cell-free nucleic acids are DNA. It should be noted that, term “sequencing data” used herein refers to “sequence reads”, which corresponds to nucleic acids subjected to sequencing.

In embodiments of the present disclosure, the sequencing result includes lengthen of the cell-free nucleic acids.

In embodiments of the present disclosure, the cell-free nucleic acids in the biological sample are sequenced by paired-end sequencing, single-end sequencing or single molecule sequencing. Therefore, lengthen of the cell-free nucleic acids may be obtained easily, which is conducive to subsequent steps.

S200: Determining the Number of the Cell-Free Nucleic Acids in a Length Falling into a Predetermined Range

The number of the cell-free nucleic acids in the length falling into the predetermined range in the biological sample is determined based on the sequencing result.

It should be noted that, term “length” used herein refers to “length of nucleic acid (read)” in base-pairs (bp).

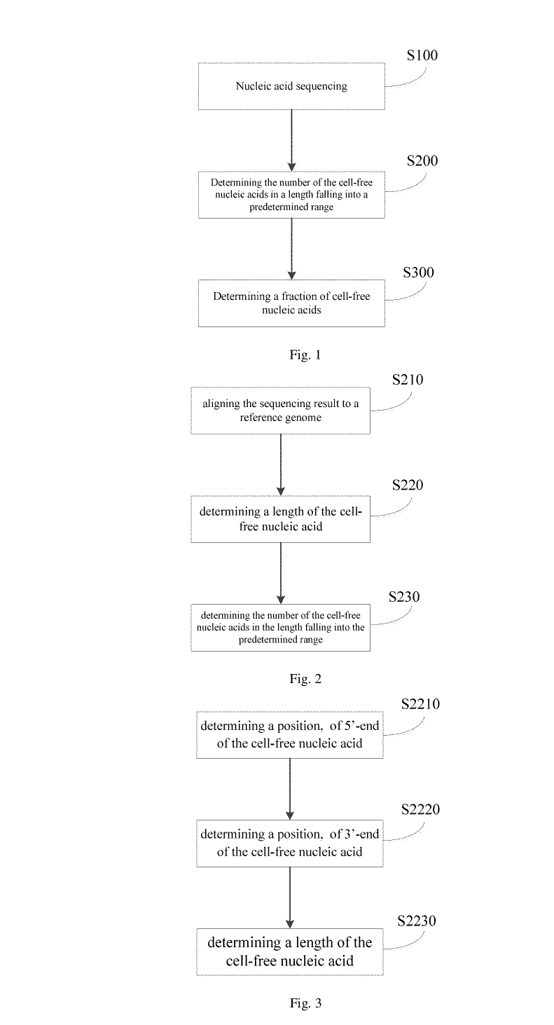

In embodiments of the present disclosure, with reference to FIG. 2, S200 further includes steps as follows:

S210: the sequencing result is aligned to a reference genome. Specifically, the sequencing result is alighed to the reference genome, so as to construct a dataset consisting of a plurality of uniquely-mapped reads, where each read in the dataset can be mapped to a position of the reference genome only; preferably, there is no mismapped read or at most one mismapped read or at most two misalighed reads.

S220: a length of the cell-free nucleic acid is determined. Specifically, a length of the cell-free nucleic acid corresponding to each uniquely-mapped read in the datasets is determined.

S230: the number of the cell-free nucleic acids falling into the predetermined range is determined. Specifically, the number of the cell-free nucleic acids in the length falling into the predetermined range is determined.

Therefore, the number of the cell-free nucleic acids in the length falling into the predetermined range in the biological sample can be determined easily, which gives rise to an accurate and reliable result and good reproducibility.

In embodiments of the present disclosure, in S220, the length of each read uniquely mapped to the reference genome is determined as the length of the cell-free nucleic acid corresponding to the read. Therefore, the length of the cell-free nucleic acid corresponding to each uniquely-mapped read in the dataset can be determined accurately.

In embodiments of the present disclosure, in the case that the cell-free nucleic acids in the biological sample are sequenced by the paired-end sequencing, with reference to FIG. 3, S220 includes the following steps.

S2210: a position, corresponding to the reference genome, of 5′-end of the cell-free nucleic acid is determined. Specifically, it is determined that the position, corresponding to the reference genome, of 5′-end of the cell-free nucleic acid based on sequencing data at one end of each uniquely-mapped read obtained in the paired-end sequencing.

S2220: a position, corresponding to the reference genome, of 3′-end of the cell-free nucleic acid is determined. Specifically, it is determined that the position, corresponding to the reference genome, of 3′-end of the cell-free nucleic acid based on sequencing data at the other end of same uniquely-mapped read obtained in the paired-end sequencing.

S2230: the length of the cell-free nucleic acid is determined. Specifically, the length of the cell-free nucleic acid is determined based on the position of 5′-end of the cell-free nucleic acid and the position of 3′-end of the cell-free nucleic acid.

Therefore, the length of the cell-free nucleic acid corresponding to each uniquely-aligned read in the dataset can be determined accurately.

S300: Determining the Fraction of the Cell-Free Nucleic Acids

It is determined that the fraction of the cell-free nucleic acids from the predetermined source in the biological sample based on the number of the cell-free nucleic acids in the length falling into the predetermined range.

Further, the method according to the present disclosure further includes determining the predetermined range (S400, not shown in Figures). In embodiments of the present disclosure, the predetermined range is determined based on a plurality of control samples, in each of which the fraction of the cell-free nucleic acids from the predetermined source is known. Therefore, the predetermined range can be determined with an accurate and reliable result. In embodiments of the present disclosure, the predetermined range is determined based on at least 20 control samples.

In embodiments of the present disclosure, with reference to FIG. 4, S400 includes the following steps.

S410: it is determined that lengths of the cell-free nucleic acids in the plurality of control samples.

S420: it is determined that a percentage of the cell-free nucleic acids present in each candidate length range. Specifically, a plurality of candidate length ranges are set, and it is determined that a percentage of the cell-free nucleic acids, obtained from each of the plurality of control samples, present in each candidate length range.

S430: a correlation coefficient is determined. Specifically, it is determined that the correlation coefficient between each candidate length range and the fraction of the cell-free nucleic acids from the predetermined source, based on the percentage of the cell-free nucleic acids, obtained from each of the plurality of control samples, present in each candidate length range and the fraction of the cell-free nucleic acids from the predetermined source in the control samples; and

S440: the predetermined range is determined. Specifically, a candidate length range with the largest correlation coefficient is determined as the predetermined range.

Therefore, the predetermined range can be determined accurately and efficiently.

In embodiments of the present disclosure, the candidate length range is of a span of 1 bp to 20 bp.

In embodiments of the present disclosure, the plurality of candidate length ranges is of a step size of 1 bp to 2 bp.

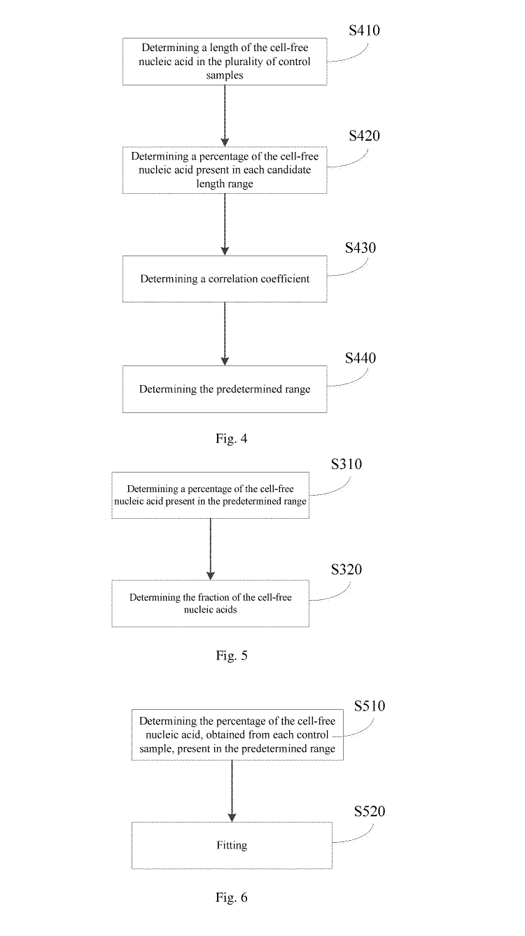

In embodiments of the present disclosure, with reference to FIG. 5, S300 further includes the following steps.

S310: a percentage of the cell-free nucleic acids present in the predetermined range is determined. Specifically, the percentage of the cell-free nucleic acids present in the predetermined range is determined based on the number of cell-free nucleic acids in the length falling into the predetermined range.

S320: the fraction of the cell-free nucleic acids from the predetermined source in the biological sample is determined. Specifically, the fraction of the cell-free nucleic acids from the predetermined source in the biological sample is determined based on the percentage of the cell-free nucleic acids present in the predetermined range, according to a predetermined function, in which the predetermined function is determined based on the plurality of control samples.

Therefore, the fraction of the cell-free nucleic acids from the predetermined source in the biological sample can be determined efficiently, which gives rise to an accurate and reliable result and good reproducibility.

In embodiments of the present disclosure, the method further includes determining the predetermined function (S500, not shown in Figures).

In some specific embodiments of the present disclosure, with reference to FIG. 6, S500 includes the following steps.

S510: it is determined that the percentage of the cell-free nucleic acids, obtained from each control sample, present in the predetermined rang.

S520: the percentage of the cell-free nucleic acids, obtained from each control sample, present in the predetermined range is fitted with the known fraction of the cell-free nucleic acid from the predetermined source to determine the predetermined function.

Therefore, the predetermined function can be determined accurately and efficiently, which is conducive to subsequent steps.

In embodiments of the present disclosure, the percentage of the cell-free nucleic acids, obtained from each control sample, present in the predetermined range is fitted with the known fraction of the cell-free nucleic acid from the predetermined source by a linear fitting.

In embodiments of the present disclosure, the cell-free nucleic acid from the predetermined source is cell-free fetal nucleic acid obtained from a peripheral blood sample of a pregnant woman, and the predetermined range is 185 bp to 204 bp. Therefore, the fraction of the cell-free nucleic acids from the predetermined source in the biological sample can be determined accurately based on the predetermined range.

In embodiments of the present disclosure, the predetermined function is d=0.0334*p+1.6657, where d represents a fraction of cell-free fetal nucleic acids, and p represents a percentage of cell-free nucleic acid present in the predetermined range. The fraction of the cell-free nucleic acids from the predetermined source in the biological sample can be efficiently determined based on the predetermined function, which gives rise to an accurate and reliable result and good reproducibility.

It should be noted that, expression “the percentage of the cell-free nucleic acids present in the predetermined range” refers to the percentage of the number of the cell-free nucleic acids distributed in a certain predetermined length range to total number of the cell-free nucleic acids in the biological sample.

In embodiments of the present disclosure, the control sample is a peripheral blood sample obtained from a pregnant woman in which the fraction of the cell-free fetal nucleic acids is known. Therefore, the predetermined range is determined accurately.

In embodiments of the present disclosure, the control sample is a peripheral blood sample obtained from a pregnant woman with a normal male fetus, in which the fraction of the cell-free fetal nucleic acids is known to be determined by chromosome Y. Therefore, the predetermined range is determined accurately.

In embodiments of the present disclosure, the control sample is a peripheral blood sample obtained from a pregnant woman with a normal fetus. Therefore, the predetermined range is determined accurately.

In embodiments of the present disclosure, the fraction of cell-free nucleic acids in the control sample is a cell-free fetal DNA fraction which is estimated by chromosome Y. Therefore, the predetermined range can be determined by efficiently utilizing the fraction of cell-free nucleic acids of the control sample, and then the number of the cell-free nucleic acids in the length falling into the predetermined range and the cell-free fetal DNA fraction in a simple obtained from a pregnant woman under detection can be further determined.

In other embodiments of the present disclosure, the method may further include the following steps.

1) Whole genome sequencing (WGS): the sample under detection is subjected to whole genome sequencing using the high-throughput platform. Cell-free fetal DNAs in plasma, which are relatively short and in which only a small amount exceeds 300 bp in length, are sequenced by single-end sequencing or paired-end sequencing as lengthen of all cell-free fetal DNAs are need to be obtained, and the entire cell-free DNA molecule is required to be sequenced if by single-ended sequencing.

2) Obtaining uniquely-mapped reads: reads of a test sample are aligned to reference genome sequence.

3) Obtaining the length of DNA corresponding to each uniquely-mapped read based on the aligning information of each uniquely-mapped read.

4) Selecting one or more ranges with high correlation: one or more ranges with high correlation is/are selected according to the length distribution of DNA molecules.

5) Obtaining a function formula: it is obtained that the function formula between a percentage of DNA present in the one or more ranges with high correlation obtained in 4) and the known cell-free fetal DNA fraction.

6) Obtaining a percentage of DNA present in one or more selected ranges, i.e. the percentage of DNA present in one or more length range.

7) Obtaining a cell-free fetal DNA fraction of the sample under detection based on the function formula and the percentage of DNA in the sample under detection present in one or more length range.

Specifically, Step 4) includes the following steps:

I. Selecting a control sample, i.e. a samle in which the cell-free fetal DNA fraction is known.

II. All the samples are subjected to WGS sequencing, and length information of DNA molecule represented by each uniquely-aligned reads is obtained by uniquely aligning information obtained by uniquely aligning the reads to a chromosome.

III. The number of DNA molecules with a length selecting from 0 bp to Mbp (M represents a maximum length value, cell-free DNA molecule may have a length up to 400 bp) is obtained for all the control samples.

IV. A plurality of window ranges (length range) are obtained through moving a window in a certain window length in accordance with a certain step size, a percentage of DNA moleculars present in each window range, i.e. a percentage of DNA moleculars present in each length range, is calculated. It should be noted that, the number of DNA moleculars present in each window range, i.e. distributed in each length range being divided by total number of DNA molecules is defined as the percentage of DNA molecular present in each window range. For example, 1 bp, 5 bp, 10 bp or 15 bp may be taken as the window and any size selected from 1 bp to the window length may be taken as the step size. Specifically, as an example, if 5 bp was taken as the window and 2 bp was taken as the step size, then distributions of DNA moleculars in [1 bp,5 bp], [2 bp,6 bp], [4 bp,8 bp], [6 bp,10 bp] and so on may be obtained. As another example, if 5 bp was taken as the window and 5 bp was taken as the step size, then distributions of DNA moleculars in [1 bp,5 bp], [6 bp,10 bp], [11 bp,15 bp] and so on may be obtained. “total number of DNA molecules” described hereinbefore refers to the total number of all DNA molecules with different length.

V. A window range or a combination of window ranges (i.e. a length range or a plurality of length ranges) in which the percentage of DNA molecules present is highly correlated with the known cell-free fetal DNA fraction is finded out, and a function formula is established.

Device for Determining a Fraction of Cell-Free Nucleic Acids in a Biological Sample

In a second aspect, the present disclosure further provides a device for determining a fraction of cell-free nucleic acids from a predetermined source in a biological sample. The inventors have surprisingly found that, the device of the present disclosure is suitable to carry out the method for determining a fraction of cell-free nucleic acids from a predetermined source in a biological sample described hereinbefore, by which the fraction of the cell-free nucleic acids from the predetermined source in the biological sample, especially the fraction of the cell-free fetal nucleic acids in a peripheral blood sample obtained from a pregnant woman, or the fraction of cell-free tumor derived nucleic acids in a peripheral blood sample obtained from a subject suffering from tumor, suspected to suffer from tumor or subjected to tumor screening can be accurately and efficiently determined.

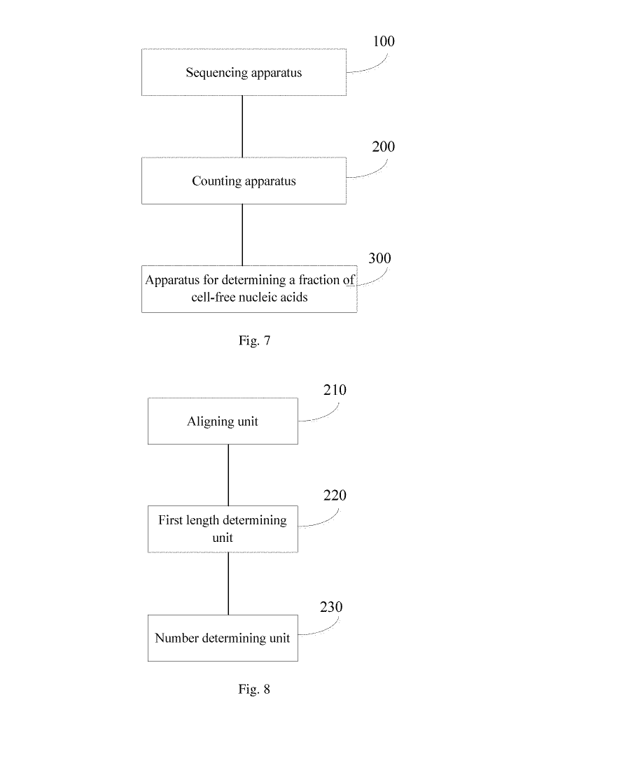

In embodiments of the present disclosure, with reference to FIG. 7, the device includes: a sequencing apparatus 100, a counting apparatus 200 and an apparatus 300 for determining a fraction of cell-free nucleic acids.

Specifically, the sequencing apparatus 100 is configured to sequence cell-free nucleic acids contained in the biological sample, so as to obtain a sequencing result consisting of a plurality of sequencing data. The counting apparatus 200 is connected to the sequencing apparatus 100 and configured to determine the number of the cell-free nucleic acids in a length falling into a predetermined range in the biological sample based on the sequencing result. The apparatus 300 for determining a fraction of cell-free nucleic acids is connected to the counting apparatus 200 and configured to determine the fraction of the cell-free nucleic acids from the predetermined source in the biological sample based on the number of the cell-free nucleic acids in the length falling into the predetermined range.

In embodiments of the present disclosure, the type of the biological sample is not particularly limited. In specific embodiments of the present disclosure, the biological sample is a peripheral blood sample. In embodiments of the present disclosure, the cell-free nucleic acid from the predetermined source is selected from one of the followings: cell-free fetal nucleic acids or cell-free maternal nucleic acids in a peripheral blood sample obtained from a pregnant woman, or cell-free tumor derived nucleic acids or cell-free non-tumor derived nucleic acids in a peripheral blood sample obtained from a subject suffering from tumor, suspected to suffer from tumor or subjected to tumor screening. Therefore, the fraction of the cell-free fetal nucleic acids in the peripheral blood sample obtained from the pregnant woman, or the fraction of cell-free tumor derived nucleic acids in the peripheral blood sample obtained from the subject suffering from tumor, suspected to suffer from tumor or subjected to tumor screening can be easily determined. In embodiments of the present disclosure, the nucleic acids are DNA.

In embodiments of the present disclosure, the sequencing result includes lengthes of the cell-free nucleic acids.

In embodiments of the present disclosure, the cell-free nucleic acids in the biological sample are sequenced by paired-end sequencing, single-end sequencing or single molecule sequencing. Therefore, lengthes of the cell-free nucleic acids may be obtained easily, which is conducive to subsequent steps.

In embodiments of the present disclosure, with reference to FIG. 8, the counting apparatus 200 further includes: an aligning unit 210, a first length determining unit 220 and a number determining unit 230. Specifically, the aligning unit 210 is configured to align the sequencing result to a reference genome, so as to construct a dataset consisting of a plurality of uniquely-mapped reads, where each read in the dataset can be mapped to a position of the reference genome only. The first length determining unit 220 is connected to the aligning unit 210 and configured to determine a length of the cell-free nucleic acid corresponding to each uniquely-mapped read in the dataset. The number determining unit 230 is connected to the first length determining unit 220 and configured to determine the number of the cell-free nucleic acids in the length falling into the predetermined range. Therefore, the number of the cell-free nucleic acids in the length falling into the predetermined range in the biological sample can be determined easily, which gives rise to an accurate and reliable result and good reproducibility.

In embodiments of the present disclosure, the first length determining apparatus 220 is configured to determine the length of each read uniquely mapped to the reference genome as the length of the cell-free nucleic acid corresponding to the read. Therefore, the length of the cell-free nucleic acid corresponding to each uniquely-mapped read in the dataset can be determined accurately.

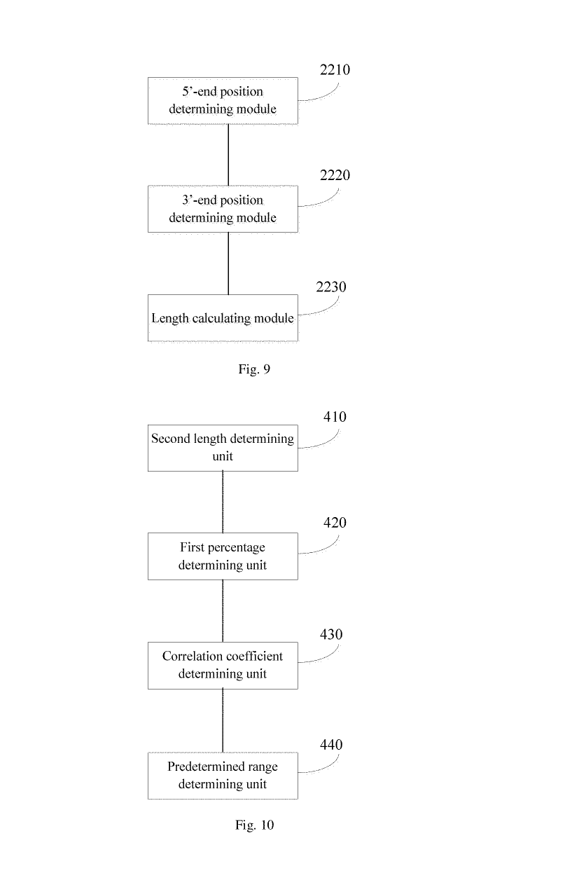

In embodiments of the present disclosure, with reference to FIG. 9, in the case that the cell-free nucleic acids in the biological sample are sequenced by the paired-end sequencing, the first length determining unit 220 further includes: a 5′-end position determining module 2210, a 3′-end position determining module 2220 and a length calculating module 2230. Specifically, the 5′-end position determining module 2210 is configured to determine a position, corresponding to the reference genome, of 5′-end of the cell-free nucleic acid, based on sequencing data at one end of each uniquely-mapped read obtained in the paired-end sequencing. The 3′-end position determining module 2220 is connected to the 5′-end position determining module 2210 and configured to determine a position, corresponding to the reference genome, of 3′-end of the cell-free nucleic acid, based on sequencing data at the other end of same uniquely-mapped read obtained in the paired-end sequencing. The length calculating module 2230 is connected to the 3′-end position determining module 2220 and configured to determine the length of the cell-free nucleic acid based on the position of 5′-end of the cell-free nucleic acid and the position of 3′-end of the cell-free nucleic acid. Therefore, the length of the cell-free nucleic acid corresponding to each uniquely-mapped read in the dataset can be determined accurately.

In embodiments of the present disclosure, the device further includes a predetermined range determining apparatus 400 configured to determine the predetermined range based on a plurality of control samples, in each of which the fraction of the cell-free nucleic acids from the predetermined source is known, optionally, the predetermined range is determined based on at least 20 control samples.

In embodiments of the present disclosure, with reference to FIG. 10, the predetermined range determining apparatus 400 further includes: a second length determining unit 410, a first Percentage determining unit 420, a correlation coefficient determining unit 430 and a predetermined range determining unit 440. Specifically, the second length determining unit 410 is configured to determine lengthen of the cell-free nucleic acids in the plurality of control samples. The first percentage determining unit 420 is connected to the second length determining unit 410 and configured to set a plurality of candidate length ranges and determine a percentage of the cell-free nucleic acids, obtained from each of the plurality of control samples, present in each candidate length range. The correlation coefficient determining unit 430 is connected to the first percentage determining unit 420 and configured to determine a correlation coefficient between each candidate length range and the fraction of the cell-free nucleic acids from the predetermined source, based on the percentage of the cell-free nucleic acids, obtained from each of the plurality of control samples, present in each candidate length range and the fraction of the cell-free nucleic acids from the predetermined source in the control samples. The predetermined range determining unit 440 is connected to the correlation coefficient determining unit 430 and configured to select a candidate length range with the largest correlation coefficient as the predetermined range. Therefore, the predetermined range can be determined accurately and efficiently.

In embodiments of the present disclosure, the candidate length range is of a span of 1 bp to 20 bp.

In embodiments of the present disclosure, the plurality of candidate length ranges is of a step size of 1 bp to 2 bp.

In embodiments of the present disclosure, with reference to FIG. 11, the apparatus for determining a fraction of cell-free nucleic acids 300 further includes: a second percentage determining unit 310 and a unit 320 for calculating a fraction of cell-free nucleic acids. Specifically, the second percentage determining unit 310 is configured to determine a percentage of the cell-free nucleic acids present in the predetermined range based on the number of cell-free nucleic acids in the length falling into the predetermined range. The unit 320 for calculating a fraction of cell-free nucleic acids is connected to the second percentage determining unit 310 and configured to determine the fraction of the cell-free nucleic acids from the predetermined source in the biological sample, based on the percentage of the cell-free nucleic acids present in the predetermined range, according to a predetermined function, in which the predetermined function is determined based on the plurality of control samples. Therefore, the fraction of the cell-free nucleic acids from the predetermined source in the biological sample can be determined efficiently, which gives rise to an accurate and reliable result and good reproducibility.

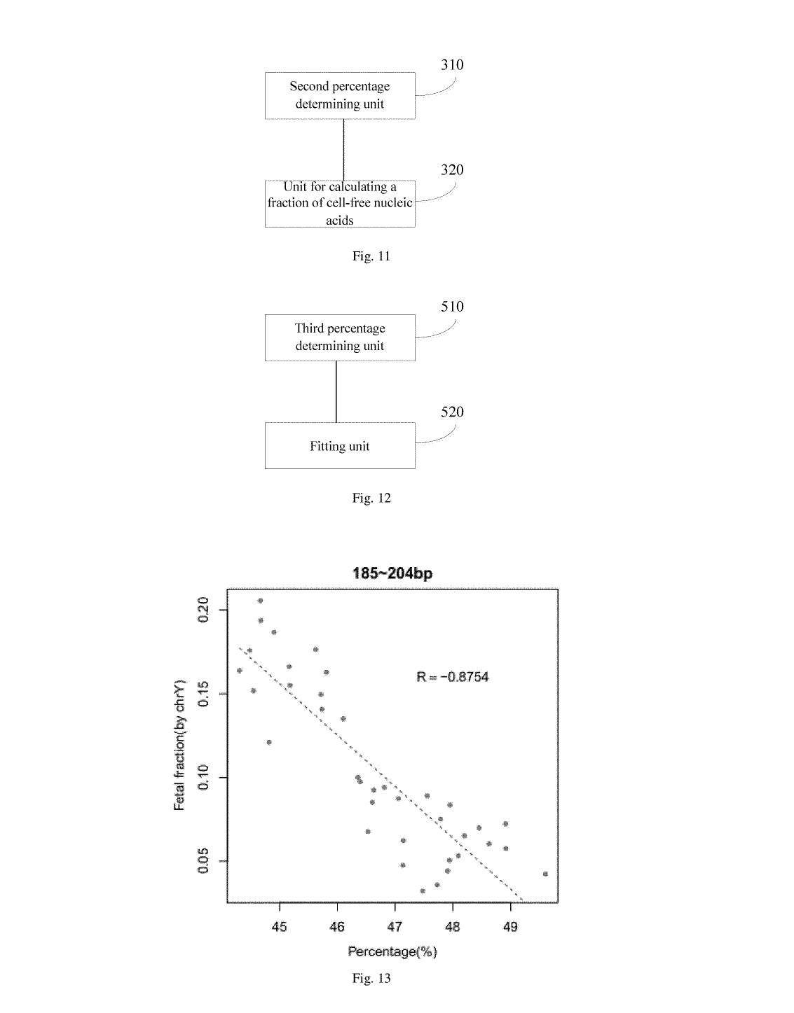

In embodiments of the present disclosure, the device further includes a predetermined function determining apparatus 500. With reference to FIG. 12, the predetermined function determining apparatus 500 includes: a third percentage determining unit 510 and a fitting unit 520. Specifically, third percentage determining unit 510 is configured to determine the percentage of the cell-free nucleic acids, obtained from each control sample, present in the predetermined range. The fitting unit 520 is connected to the third percentage determining unit 510 and configured to fit the percentage of the cell-free nucleic acids, obtained from each control sample, present in the predetermined range, with the known fraction of the cell-free nucleic acid from the predetermined source, to determine the predetermined function. Therefore, the predetermined function can be determined accurately and reliably, which is conducive to subsequent steps. In embodiments of the present disclosure, the percentage of the cell-free nucleic acids, obtained from each control sample, present in the predetermined range is fitted with the known fraction of the cell-free nucleic acid from the predetermined source by a linear fitting.

In embodiments of the present disclosure, the cell-free nucleic acid from the predetermined source is cell-free fetal nucleic acid obtained from a peripheral blood sample of a pregnant woman, and the predetermined range is 185 bp to 204 bp. Therefore, the fraction of the cell-free nucleic acids from the predetermined source in the biological sample can be determined accurately based on the predetermined range.

In embodiments of the present disclosure, the predetermined function is d=0.0334*p+1.6657, where d represents a fraction of cell-free fetal nucleic acids, and p represents a percentage of cell-free nucleic acid present in the predetermined range. The fraction of the cell-free nucleic acids from the predetermined source in the biological sample can be efficiently determined based on the predetermined function, which gives rise to an accurate and reliable result and good reproducibility.

In embodiments of the present disclosure, the control sample is a peripheral blood sample obtained from a pregnant woman in which the fraction of the cell-free fetal nucleic acids is known.

In embodiments of the present disclosure, the control sample is a peripheral blood sample obtained from a pregnant woman with a normal male fetus, in which the fraction of the cell-free fetal nucleic acids is known to be determined by chromosome Y. Therefore, the predetermined range is determined accurately.

In embodiments of the present disclosure, the control sample is a peripheral blood sample obtained from a pregnant woman with a normal male fetus. Therefore, the predetermined range is determined accurately.

In embodiments of the present disclosure, the fraction of cell-free nucleic acids in the control sample is a cell-free fetal DNA fraction which is determined by a device suitable for estimation with chromosome Y. Therefore, the predetermined range can be determined by efficiently utilizing the fraction of cell-free nucleic acids of the control sample, and then the number of the cell-free nucleic acids in the length falling into the predetermined range and the cell-free fetal DNA fraction in a simple obtained from a pregnant woman under detection can be further determined.

Method and System for Determining Sexuality of Twins

In a third aspect, the present disclosure provides a method for determining sexuality of twins. In embodiments of the present disclosure, the method includes: performing sequencing on cell-free nucleic acids contained in a peripheral blood sample obtained from a pregnant woman with twins, so as to obtain a sequencing result consisting of a plurality of sequencing data; determining a first cell-free fetal DNA fraction based on the sequencing data, by the method hereinbefore for determining the fraction of cell-free nucleic acids in a biological sample; determining a second cell-free fetal DNA fraction based on a sequencing data derived from chromosome Y in the sequencing result; and determining the sexuality of the twins based on the first cell-free fetal DNA fraction and the second cell-free fetal DNA fraction. The inventors have surprisingly found that, sexuality of twins in a pregnant woman can be ccurately and efficiently determined by the method of the present disclosure.

In embodiments of the present disclosure, the second cell-free fetal DNA fraction is determined according to the following formula:

fra.chry=(chry.ER %−Female.chry.ER %)/(Man.chry.ER %−Female.chry.ER %)*100%,

where fra.chry represents the second cell-free fetal DNA fraction, chry.ER % represents a percentage of the sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man to total sequencing data thereof.

Therefore, the second cell-free fetal DNA fraction can be determined accurately.

In embodiments of the present disclosure, determining the sexuality of the twins based on the first cell-free fetal DNA fraction and the second cell-free fetal DNA fraction further includes: (a) determining a ratio of the second cell-free fetal DNA fraction to the first cell-free fetal DNA fraction; and (b) determining the sexuality of the twins by comparing the ratio determined in (a) with a first threshold and a second threshold predetermined. Therefore, the sexuality of the twins can be determined efficiently.

In embodiments of the present disclosure, the first threshold is determined based on a pluratity of control samples obtained from pregnant women known with female twins, and the second threshold is determined based on a pluratity of control samples obtained from pregnant women known with male twins.

In embodiments of the present disclosure, both fetuses of the twins are female if the ratio of the second cell-free fetal DNA fraction to the first cell-free fetal DNA fraction is lower than the first threshold, both fetuses of the twins are male if the ratio of the second cell-free fetal DNA fraction to the first cell-free fetal DNA fraction is greater than the second threshold, and the twins include a male fetus and a female fetus if the ratio of the second cell-free fetal DNA fraction to the first cell-free fetal DNA fraction is equal to the first threshold or the second threshold, or between the first threshold and the second threshold.

In embodiments of the present disclosure, the first threshold is 0.35, and the second threshold is 0.7.

In a fourth aspect, the present disclosure provides a system for determining sexuality of twins. In embodiments of the present disclosure, the system includes:

a first cell-free fetal DNA fraction determining device, being the device hereinbefore for determining the fraction of cell-free nucleic acids in the biological sample, and configured to sequence cell-free nucleic acids contained in a peripheral blood sample obtained from a pregnant woman with twins, so as to obtain a sequencing result consisting of a plurality of sequencing data, and configured to determine a first cell-free fetal DNA fraction based on the sequencing data;

a second cell-free fetal DNA fraction determining device, configured to determine a second cell-free fetal DNA fraction based on a sequencing data derived from chromosome Y in the sequencing result; and

a sexuality determining device, configured to determine the sexuality of the twins based on the first cell-free fetal DNA fraction and the second cell-free fetal DNA fraction.

The inventors have surprisingly found that, sexuality of twins in a pregnant woman can be ccurately and efficiently determined by the system of the present disclosure.

In embodiments of the present disclosure, the second cell-free fetal DNA fraction is determined according to the following formula:

fra.chry=(chry.ER %−Female.chry.ER %)/(Man.chry.ER %−Female.ch.ER %)*100%,

where fra.chry represents the second cell-free fetal DNA fraction, chry.ER % represents a percentage of the sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man to total sequencing data thereof. Therefore, the second cell-free fetal DNA fraction can be determined accurately.

In embodiments of the present disclosure, the sexuality determining device further includes: a ratio determining unit, configured to determine a ratio of the second cell-free fetal DNA fraction to the first cell-free fetal DNA fraction; and a comparison unit, configured to compare the ratio determined by the ratio determining unit with a first threshold and a second threshold predetermined, so as to determine the sexuality of the twins. Therefore, the sexuality of the twins can be determined efficiently.

In embodiments of the present disclosure, the first threshold is determined based on a pluratity of control samples obtained from pregnant women known with female twins, and the second threshold is determined based on a pluratity of control samples obtained from pregnant women known with male twins.

In embodiments of the present disclosure, both fetuses of the twins are female if the ratio of the second cell-free fetal DNA fraction to the first cell-free fetal DNA fraction is lower than the first threshold, both fetuses of the twins are male if the ratio of the second cell-free fetal DNA fraction to the first cell-free fetal DNA fraction is greater than the second threshold, and the twins include a male fetus and a female fetus if the ratio of the second cell-free fetal DNA fraction to the first cell-free fetal DNA fraction is equal to the first threshold or the second threshold, or between the first threshold and the second threshold.

In embodiments of the present disclosure, the first threshold is 0.35, and the second threshold is 0.7.

Method and System for Detecting a Chromosome Aneuploidy of Twins

In a fifth aspect, the present disclosure provides a method for detecting a chromosome aneuploidy of twins. In embodiments of the present disclosure, the method includes:

performing sequencing on cell-free nucleic acids contained in a peripheral blood sample obtained from a pregnant woman with twins, so as to obtain a sequencing result consisting of a plurality of sequencing data;

determining a first cell-free fetal DNA fraction, based on the sequencing data, by the method hereinbefore for determining the fraction of cell-free nucleic acids in a biological sample; or estimating a fetal fraction by chromosome Y (fra.chrY %) as the first cell-free fetal DNA fraction according to the following formula:

fra.chry=(chry.ER %—Female.chr.ER %)/(Man.chry.UR %−Female.chry.ER %)*100%,

where fra.chry represents the first cell-free fetal DNA fraction, chry.ER % represents a percentage of sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;

determining a third cell-free fetal DNA fraction, based on a sequencing data derived from a predetermined chromosome in the sequencing result; and

determining whether the twins under detection have aneuploidy with respect to the predetermined chromosome based on the first cell-free fetal DNA fraction and the third cell-free fetal DNA fraction.

Therefore, the chromosome aneuploidy of twins can be detected acurately and efficiently.

In embodiments of the present disclosure, the third cell-free fetal DNA fraction is determined according to the following formula:

fra.chri=2*(chri.ER %/adjust.chri.ER %−1)*100%,

where fra.chri represents the third cell free fetal DNA fraction, i represents a serial number of the predetermined chromosome, and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with normal twins to total sequencing data thereof. Therefore, the third cell-free fetal DNA fraction can be determined accurately.

In embodiments of the present disclosure, determining whether the twins under detection have aneuploidy with respect to the predetermined chromosome based on the first cell-free fetal DNA fraction and the third cell-free fetal DNA fraction further includes: (a) determining a ratio of the third cell-free fetal DNA fraction to the first cell-free fetal DNA fraction; and (b) determining whether the twins under detection have aneuploidy with respect to the predetermined chromosome by comparing the ratio determined in (a) with a third threshold and a fourth threshold predetermined. Therefore, the chromosome aneuploidy of twins can be detected efficiently.

In embodiments of the present disclosure, the third threshold is determined based on a pluratity of control samples obtained from pregnant women with twins known not to have aneuploidy with respect to the predetermined chromosome, and the fourth threshold is determined based on a pluratity of control samples obtained from pregnant women with twins known to have aneuploidy with respect to the predetermined chromosome.

In embodiments of the present disclosure, both fetuses of the twins have no aneuploidy with respect to the predetermined chromosome if the ratio of the third cell-free fetal DNA fraction to the first cell-free fetal DNA fraction is lower than the third threshold, both fetuses of the twins have aneuploidy with respect to the predetermined chromosome if the ratio of the third cell-free fetal DNA fraction to the first cell-free fetal DNA fraction is greater than the fourth threshold, and one fetus of the twins has the aneuploidy with respect to the predetermined chromosome, while the other fetus of the twins has no aneuploidy with respect to the predetermined chromosome if the ratio of the third cell-free fetal DNA fraction to the first cell-free fetal DNA fraction is equal to the third threshold or the fourth threshold, or between the third threshold and the fourth threshold.

In embodiments of the present disclosure, the third threshold is 0.35, and the fourth threshold is 0.7.

In embodiments of the present disclosure, the predetermined chromosome is at least one selected from chromosomes 18, 21 and 23.

In a sixth aspect, the present disclosure provides a system for determining a chromosome aneuploidy of twins. In embodiments of the present disclosure, the system includes:

a first cell-free fetal DNA fraction determining device, being the device hereinbefore for determining the fraction of cell-free nucleic acids in the biological sample, and configured to sequence cell-free nucleic acids contained in a peripheral blood sample obtained from a pregnant woman with twins, so as to obtain a sequencing result consisting of a plurality of sequencing data, and configured to determine a first cell-free fetal DNA fraction based on the sequencing data or estimate a fetal fraction by chromosome Y (fra.chrY %) as the first cell-free fetal DNA fraction according to the following formula:

fra.chry=(chry.ER %−Female.chr.ER %)/(Man.chry.UR %−Female.chry.ER %)*100%, where

fra.chry represents the first cell-free fetal DNA fraction, chry.ER % represents a percentage of sequencing data derived from chromosome Y in the sequencing result to total sequencing data; Female.chry.ER % represents an average percentage of sequencing data of cell-free nucleic acids derieved from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man perdetermined to total sequencing data thereof;

a third cell-free fetal DNA fraction determining device, configured to determine a third cell-free fetal DNA fraction based on a sequencing data derived from a predetermined chromosome in the sequencing result; and a first aneuploidy determining device, configured to determine whether the twins under detection have aneuploidy with respect to the predetermined chromosome based on the first cell-free fetal DNA fraction and the third cell-free fetal DNA fraction. The inventors have surprisingly found that, the chromosome aneuploidy of twins can be detected ccurately and efficiently by the system according to the present disclosure.

In embodiments of the present disclosure, the third cell-free fetal DNA fraction is determined according to the following formula:

fra.chri=2*(chri.ER %/adjust.chri.ER %−1)*100% where fra.chri represents the third cell free fetal DNA fraction, i represents a serial number of the predetermined chromosome, and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with normal twins to total sequencing data thereof. Therefore, the third cell-free fetal DNA fraction can be determined accurately.

In embodiments of the present disclosure, the first aneuploidy determining device further includes:

a ratio determining unit, configured to determine a ratio of the third cell-free fetal DNA fraction to the first cell-free fetal DNA fraction; and

a comparison unit, configured to compare the ratio determined by the ratio determining unit with a third threshold and a fourth threshold predetermined, so as to determine whether the twins under detection have aneuploidy with respect to the predetermined chromosome. Therefore, the chromosome aneuploidy of twins can be detected efficiently.

In embodiments of the present disclosure, the third threshold is determined based on a pluratity of control samples obtained from pregnant women with twins known not to have aneuploidy with respect to the predetermined chromosome, and the fourth threshold is determined based on a pluratity of control samples obtained from pregnant women with twins known to have aneuploidy with respect to the predetermined chromosome.

In embodiments of the present disclosure, both fetuses of the twins have no aneuploidy with respect to the predetermined chromosome if the ratio of the third cell-free fetal DNA fraction to the first cell-free fetal DNA fraction is lower than the third threshold, both fetuses of the twins have aneuploidy with respect to the predetermined chromosome if the ratio of the third cell-free fetal DNA fraction to the first cell-free fetal DNA fraction is greater than the fourth threshold, and one fetus of the twins has the aneuploidy with respect to the predetermined chromosome, while the other fetus of the twins has no aneuploidy with respect to the predetermined chromosome if the ratio of the third cell-free fetal DNA fraction to the first cell-free fetal DNA fraction is equal to the third threshold or the fourth threshold, or between the third threshold and the fourth threshold. Therefore, the chromosome aneuploidy of twins can be detected efficiently.

In embodiments of the present disclosure, the third threshold is 0.35, and the fourth threshold is 0.7.

In embodiments of the present disclosure, the predetermined chromosome is at least one selected from chromosomes 18, 21 and 23.

In a seventh aspect, the present disclosure provides a method for determining a chromosome aneuploidy of twins. In embodiments of the present disclosure, the method includes:

performing sequencing on cell-free nucleic acids contained in a peripheral blood sample obtained from a pregnant woman with twins, so as to obtain a sequencing result consisting of a plurality of sequencing data;

determining a fraction xᵢ, of the number of sequencing data derived from chromosome i in the sequencing result to total sequencing data, where i represents a serial number of the chromosome, and i is any integer in the range of 1 to 22;

determining a T score of the chromosome i according to Tᵢ(xᵢ=μᵢ)/σᵢ, where i represents the serial number of the chromosome and is any integer in the range of 1 to 22, μᵢ represents an average percentage of sequencing data of the chromosome i selected as a reference system in a reference database to total sequencing data thereof, σᵢ represents a standard deviation of percentages of the sequencing data of the chromosome i selected as the reference system in the reference database to total sequencing data thereof,

determining an L score of the chromosome i according to Lᵢ=log(d(Tᵢ, a))/log(d(T2ᵢ,a)), where i represents the serial number of the chromosome and i is any integer in the range of 1 to 22, T2ᵢ=(xᵢ−μᵢ*(1+fra/2))/σᵢ; d(Tᵢ, a) and d(T2ᵢ, a) represent t distribution probability density function, where a represents degree of freedom, fra represents a cell-free fetal DNA fraction determined by the method hereinbefore for determining the fraction of cell-free nucleic acids in a biological sample or a fetal fraction estimated by chromosome Y (fra.chrY %), fra.chry=(chry.ER %−Female.chr.ER %)/(Man.chry.UR %−Female.chry.ER %)*100%, where fra.chry represents a cell-free fetal DNA fraction, chry.ER % represents a percentage of sequencing data derived from chromosome Y in the sequencing result to said total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;

plotting a four-quadrant diagram with T as vertical coordinate and L as horizontal coordinate by zoning with a first straight line where T=predetermined fifth threshold and a second straight line where L=predetermined sixth threshold, wherein both fetuses of the twins are determined to have trisome if a sample under detection is determined to be of the T score and the L score falling into a first quadrant; one fetus of the twins is determined to be of trisome and the other fetus of the twins is determined to be normal if a sample under detection is determined to be of the T score and the L score falling into a second quadrant; both fetuses of the twins are determined to be normal if a sample under detection is determined to be of the T score and the L score falling into a third quadrant; the twins are determined to have a low fetal fraction if a sample under detection is determined to be of the T score and the L score falling into a fourth quadrant, such a result is not adopted. The inventors have surprisingly found that, the detection of the chromosome aneuploidy of twins of a pregnant woman and the determination of whether the twins under detection have aneuploidy with respect to the predetermined chromosome can be achieved accurately and efficiently by the method for determining the chromosome aneuploidy of twins according to the present disclosure.

In an eighth aspect, the present disclosure provides a system for determining a chromosome aneuploidy of twins. In embodiments of the present disclosure, the system includes:

an xᵢ value determining device, configured to sequence cell-free nucleic acids contained in a peripheral blood sample obtained from a pregnant woman with twins, so as to obtain a sequencing result consisting of a plurality of sequencing data, and configured to determine a fraction xᵢ of the number of sequencing data derived from chromosome i in the sequencing result to total sequencing data, where i represents a serial number of the chromosome and i is any integer in the range of 1 to 22;

a T score determining device, configured to determine a T score of the chromosome i according to Tᵢ=(xᵢ−μᵢ)/σᵢ, where i represents the serial number of the chromosome and i is any integer in the range of 1 to 22, μᵢ represents an average percentage of sequencing data of the chromosome i selected as a reference system in a reference database to total sequencing data thereof, σᵢ represents a standard deviation of percentages of the sequencing data of the chromosome i selected as the reference system in the reference database to total sequencing data thereof,

an L score determining device, configured to determine an L score of the chromosome i according to Lᵢ=log(d(Tᵢ, a))/log(d(T2ᵢ,a)), where i represents the serial number of the chromosome and i is any integer in the range of 1 to 22, T2ᵢ=(xᵢ−μᵢ*(1+fra/2))/σᵢ; d(Tᵢ, a) and d(T2₁, a) represent t distribution probability density function, where a represents degree of freedom, fra represents a first cell-free fetal DNA fraction determined by the method hereinbefore or a fetal fraction estimated by chromosome Y (fra.chrY %), fra.chry=(chry.ER %−Female.chr.ER %)/(Man.chry.UR %−Female.chry.ER %)*100%, where fra.chry represents a second cell-free fetal DNA fraction, chry.ER % represents a percentage of sequencing data derived from chromosome Y in the sequencing result to said total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;

a second aneuploidy determining device, configured to plot a four-quadrant diagram with T as vertical coordinate and L as horizontal coordinate by zoning with a first straight line where T=predetermined fifth threshold and a second straight line where L=predetermined sixth threshold, wherein both fetuses of the twins are determined to have trisome if a sample under detection is determined to be of the T score and the L score falling into a first quadrant; one fetus of the twins is determined to have trisome and the other fetus of the twins is determined to be normal if a sample under detection is determined to be of the T score and the L score falling into a second quadrant; both fetuses of the twins are determined to be normal if a sample under detection is determined to be of the T score and the L score falling into a third quadrant; the twins are determined to have a low fetal fraction if a sample under detection is determined to be of the T score and the L score falling into a fourth quadrant, such a result is not adopted. The inventors have surprisingly found that, the detection of the chromosome aneuploidy of twins of a pregnant woman and the determination of whether the twins under detection have aneuploidy with respect to the predetermined chromosome can be achieved accurately and efficiently by the method for determining the chromosome aneuploidy of twins according to the present disclosure.

It should be noted that, “reference database” described in “μᵢ represents an average value of percentages of sequencing data of the chromosome i selected as a reference system in a reference database to total sequencing data thereof” refers to cell-free nucleic acids in a peripheral blood sample obtained from a pregnant woman with a normal fetus (female fetus, male fetus, single fetus or twins) or sequencing data (reads).

The “sequencing data” expressed in “sequencing data derived from chromosome Y” means reads obtained in sequencing.

In some specific embodiments of the present disclosure, terms “xᵢ”, “ERi” and “Chri.ER %” are changeable herein, that is, xᵢ may be a result obtained after subjected to GC correction. Specifically, UR and GC contents of each chromosome can be fitted by using known data obtained from normal samples to obtain a relation formula, i.e. ERᵢ=fᵢ(GCᵢ)+εᵢ. A mean value E̅R̅ᵢ of UR is calculated. For a sample to be analyzed, an ER value after correction is calculated according to the following formula and based on the above relation formula and ER and GC of the sample.

ERᵢⱼ=ERᵢ+εᵢ=ERᵢ+ERᵢⱼ−fᵢ(GCᵢⱼ)

Method and System for Detecting Fetal Chimera

In a ninth aspect, the present disclosure provides a method for detecting fetal chimera. In embodiments of the present disclosure, the method includes:

performing sequencing oncell-free nucleic acids contained in a peripheral blood sample obtained from a pregnant woman with a fetus, optionally a male fetus, so as to obtain a sequencing result consisting of a plurality of sequencing data;

determining a first cell-free fetal DNA fraction, based on the sequencing data, by the method hereinbefore, or estimating a fetal fraction by chromosome Y (fra.chrY %) as the first cell-free fetal DNA fraction according to the following formula:

fra.chry=(chry.ER %−Female.chr.ER %)/(Man.chry.UR %−Female.chry.ER %)*100%,

where fra.chry represents the first cell-free fetal DNA fraction, chry.ER % represents a percentage of sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;

determining a third cell-free fetal DNA fraction based on sequencing data derived from a predetermined chromosome in the sequencing result; and

determining whether the fetus under detection has fetal chimera with respect to the predetermined chromosome based on the first cell-free fetal DNA fraction and the third cell-free fetal DNA fraction.

Therefore, whether the fetus under detection has fetal chimera with respect to a specific chromosome can be analyzed accurately.

In embodiments of the present disclosure, the method may further have the following additional technical features.

In embodiments of the present disclosure, the third cell-free fetal DNA fraction is determined by the following formula:

fra.chri=2*(chri.ER %/adjust.chri.ER %−1)*100%,

where fra.chri represents the third cell-free fetal DNA fraction, i represents a serial number of the predetermined chromosome and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with a normal fetus to total sequencing data thereof. Therefore, whether the fetus under detection has fetal chimera with respect to the specific chromosome can be analyzed with further improved efficiency.

In embodiments of the present disclosure, determining whether the fetus under detection has fetal chimera with respect to the predetermined chromosome based on the first cell-free fetal DNA fraction and the third cell-free fetal DNA fraction further includes: (a) determining a ratio of the third cell-free fetal DNA fraction to the first cell-free fetal DNA fraction; and (b) determining whether the fetus under detection has chimera with respect to the predetermined chromosome by comparing the ratio determined in (a) with a plurality of predetermined thresholds. Therefore, whether the fetus under detection has fetal chimera with respect to a specific chromosome can be analyzed with further improved efficiency.

In embodiments of the present disclosure, the plurality of predetermined thresholds includes at least one selected from:

a seventh threshold, determined based on a pluratity of control samples with the predetermined chromosome known to be of complete monosome,

an eighth threshold, determined based on a pluratity of control samples with the predetermined chromosome known to be of monosome chimera,

a ninth threshold, determined based on a pluratity of control samples with the predetermined chromosome known to be normal,

a tenth threshold, determined based on a pluratity of control samples with the predetermined chromosome known to be of complete trisome.

In embodiments of the present disclosure, the predetermined chromosome of the fetus under detection is of complete monosome, if the ratio of the third cell-free fetal DNA fraction to the first cell-free fetal DNA fraction is lower than the seventh threshold;

the predetermined chromosome of the fetus under detection is of monosome chimera, if the ratio of the third cell-free fetal DNA fraction to the first cell-free fetal DNA fraction is not lower than the seventh threshold and not greater than the eighth threshold;

the predetermined chromosome of the fetus under detection is normal, if the ratio of the third cell-free fetal DNA fraction to the first cell-free fetal DNA fraction is greater than the eighth threshold and lower than the ninth threshold;

the predetermined chromosome of the fetus under detection is of trisome chimera, if the ratio of the third cell-free fetal DNA fraction to the first cell-free fetal DNA fraction is not lower than the ninth threshold and not greater than the tenth threshold; and

the predetermined chromosome of the fetus under detection is of complete trisome, if the ratio of the third cell-free fetal DNA fraction to the first cell-free fetal DNA fraction is greater than the tenth threshold.

In embodiments of the present disclosure, the seventh threshold at least is −1 and lower than 0, optionally is −0.85;

the eighth threshold is greater than the seventh threshold and lower than 0, optionally is −0.3;

the ninth threshold is greater than 0 and lower than 1, optionally is 0.3;

the tenth threshold is greater than the ninth threshold and lower than 1, optionally is 0.85. Therefore, whether the fetus under detection has fetal chimera with respect to a specific chromosome can be analyzed with further improved efficiency.

In a tenth aspect, the present disclosure provides a system for detecting fetal chimer. In embodiments of the present disclosure, the system includes:

a first cell-free fetal DNA fraction determining device, being the device hereinbefore for determining the fraction of cell-free nucleic acids in the biological sample, and configured to sequence cell-free nucleic acids contained in a peripheral blood sample obtained from a pregnant woman with fetus, so as to obtain a sequencing result consisting of a plurality of sequencing data, and configured to determine a first cell-free fetal DNA fraction based on the sequencing data, or configured to estimate a fetal fraction by chromosome Y (fra.chrY %) as the first cell-free fetal DNA fraction according to the following formula:

fra.chry=(chry.ER %−Female.chr.ER %)/(Man.chry.UR %−Female.chry.ER %)*100%,

where fra.chry represents the first cell-free fetal DNA fraction, chry.ER % represents a percentage of sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;

a third cell-free fetal DNA fraction determining device, configured to determine a third cell-free fetal DNA fraction based on sequencing data derived from a predetermined chromosome in the sequencing result; and

a chimera determining device, configured to determine whether the fetus under detection has fetal chimera with respect to the predetermined chromosome based on the first cell-free fetal DNA fraction and the third cell-free fetal DNA fraction.

According to embodiments of the present disclosure, the method hereinbefore for determining fetal chimera can be efficiently carried out by the system above, such that whether the fetus under detection has fetal chimera can be efficiently analyzed.

In embodiments of the present disclosure, the system above for detecting fetal chimera may further include the following additional technical features.

In embodiments of the present disclosure, the third cell-free fetal DNA fraction is determined by the following formula:

fra.chri=2*(chri.ER %/adjust.chri.ER %−1)*100%,

where fra.chri represents the third cell free fetal DNA fraction, i represents a serial number of the predetermined chromosome and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with a normal fetus to total sequencing data thereof. Therefore, whether the fetus under detection has fetal chimera with respect to a specific chromosome can be analyzed with further improved efficiency.

In embodiments of the present disclosure, the chimera determining device includes:

a ratio determining unit, configured to determine a ratio of the third cell-free fetal DNA fraction to the first cell-free fetal DNA fraction; and

a comparison unit, configured to compare the ratio determined by the ratio determining unit with a plurality of predetermined thresholds, so as to determine whether the fetus under detection has chimera with respect to the predetermined chromosome.

Therefore, whether the fetus under detection has fetal chimera with respect to a specific chromosome can be analyzed with further improved efficiency.

In embodiments of the present disclosure, the plurality of predetermined thresholds includes at least one selected from:

a seventh threshold, determined based on a pluratity of control samples with the predetermined chromosome known to be of complete monosome,

an eighth threshold, determined based on a pluratity of control samples with the predetermined chromosome known to be of monosome chimera,

a ninth threshold, determined based on a pluratity of control samples with the predetermined chromosome known to be normal,

a tenth threshold, determined based on a pluratity of control samples with the predetermined chromosome known to be of complete trisome,

In embodiments of the present disclosure, the predetermined chromosome of the fetus under detection is of complete monosome, if the ratio of the third cell-free fetal DNA fraction to the first cell-free fetal DNA fraction is lower than the seventh threshold;

the predetermined chromosome of the fetus under detection is of monosome chimera, if the ratio of the third cell-free fetal DNA fraction to the first cell-free fetal DNA fraction is not lower than the seventh threshold and not greater than the eighth threshold;

the predetermined chromosome of the fetus under detection is normal, if the ratio of the third cell-free fetal DNA fraction to the first cell-free fetal DNA fraction is greater than the eighth threshold and lower than the ninth threshold;

the predetermined chromosome of the fetus under detection is of trisome chimera, if the ratio of the third cell-free fetal DNA fraction to the first cell-free fetal DNA fraction is not lower than the ninth threshold and not greater than the tenth threshold; and

the predetermined chromosome of the fetus under detection is of complete trisome, if the ratio of the third cell-free fetal DNA fraction to the first cell-free fetal DNA fraction is greater than the tenth threshold.

In embodiments of the present disclosure, the seventh threshold is greater than −1 and lower than 0, optionally is −0.85;

the eighth threshold is greater than the seventh threshold and lower than 0, optionally is −0.3;

the ninth threshold is greater than 0 and lower than 1, optionally is 0.3;

the tenth threshold is greater than the ninth threshold and lower than 1, optionally is 0.85. Therefore, whether the fetus under detection has fetal chimera with respect to a specific chromosome can be analyzed with further improved efficiency.

In an eleventh aspect, the present disclosure provides a method for detecting fetal chimera. In embodiments of the present disclosure, the method includes:

performing sequencing on cell-free nucleic acids contained in a peripheral blood sample obtained from a pregnant woman with a fetus, so as to obtain a sequencing result consisting of a plurality of sequencing data;

determining a fraction xᵢ, of the number of sequencing data derived from chromosome i in the sequencing result to total sequencing data, where i represents a serial number of the chromosome, and i is any integer in the range of 1 to 22;

determining a T score of the chromosome i according to Tᵢ=(xᵢ−μᵢ)/σᵢ, where i represents the serial number of the chromosome and i is any integer in the range of 1 to 22, μᵢ represents an average value of percentages of sequencing data of the chromosome i selected as a reference system in a reference database to total sequencing data thereof, σᵢ represents a standard deviation of percentages of the sequencing data of the chromosome i selected as the reference system in the reference database to total sequencing data thereof;

determining an L score of the chromosome i according to Lᵢ=log(d(Tᵢ, a))/log(d(T2ᵢ,a)), where i represents the serial number of the chromosome and i is any integer in the range of 1 to 22, T2ᵢ=(xᵢ−μᵢ*(1+fra/2))/σᵢ; d(Tᵢ, a) and d(T2ᵢ, a) represent t distribution probability density function, a represents degree of freedom, fra represents a cell-free fetal DNA fraction determined by the method hereinbefore for determining the fraction of cell-free nucleic acids in the biological sample;

plotting a four-quadrant diagram with T as vertical coordinate and L as horizontal coordinate by zoning with a third straight line where T=predetermined eleventh threshold and a fourth straight line where L=predetermined twelfth threshold, when the T score is not greater than 0,

wherein the fetus is determined to have complete monosome or monosome chimera with respect to the predetermined chromosome, if a sample under detection is determined to be of the T score and the L score falling into a first quadrant;

the fetus is determined to have monosome chimera with respect to the predetermined chromosome, if a sample under detection is determined to be of the T score and the L score falling into a second quadrant;

the fetus is determined to be normal with respect to the predetermined chromosome, if a sample under detection is determined to be of the T score and the L score falling into a third quadrant;

the fetus is determined to have a low fetal fraction if a sample under detection is determined to be of the T score and the L score falling into a fourth quadrant, such a result is not adopted,

plotting a four-quadrant diagram with T as vertical coordinate and L as horizontal coordinate by zoning with a fifth straight line where T=predetermined thirteenth threshold and a sixth straight line where L=predetermined fourteenth threshold, when the T score is greater than 0,

wherein the fetus is determined to have complete trisome or trisome chimera with respect to the predetermined chromosome, if a sample under detection is determined to be of the T score and the L score falling into a first quadrant;

the fetus is determined to have trisome chimera with respect to the predetermined chromosome, if a sample under detection is determined to be of the T score and the L score falling into a second quadrant;

the fetus is determined to be normal with respect to the predetermined chromosome, if a sample under detection is determined to be of the T score and the L score falling into a third quadrant;

the fetus is determined to have a low fetal fraction if a sample under detection is determined to be of the T score and the L score falling into a fourth quadrant, such a result is not adopted.

Optionally, the eleventh threshold and the thirteenth threshold each independently is 3, and the twelfth threshold and the fourteenth threshold each independently is 1.

Therefore, situations on the fetal chimera can be analyzed efficiently.

It should be noted that, the expression “normal male fetus/female fetus/fetus” means that the chromosome of the fetus is normal, for example, “normal male fetus” refers to a male fetus with normal chromosomes. Moreover, “normal male fetus/female fetus/fetus” may refer to a single fetus or twins, for example, “normal male fetus” may be normal single fetus or normal twins; “normal fetus” neither limits the sexuality of the fetus nor limits the fetus being single fetus or twins.

Embodiments of the present disclosure will be described in detail below with reference to examples, but it should be appreciated to those skilled in the art that the following examples are merely used to illustrate the present disclosure, thus shall not be construed to limit the scope of the present disclosure. An example in which the specific condition is not specified will be carried out under normal condition or condition recommended by the manufacturer. Reagents or instruments not specify the manufacturers are conventional products available from the market.

### Example 1

Cell-free fetal DNA fractions in plasma samples obtained from 11 pregnant women under detection are estimated according to the method for determining a fraction of cell-free nucleic acids from a predetermined source in a biological sample of the present disclosure, as follows.

1) Sample Collection and Treatment

2 ml peripheral blood, extracted during pregnancy from each of 11 pregnant women under detection and 37 pregnant women known with male fetuses, was subjected to plasma separation, so as to obtain a peripheral blood sample of each pregnant woman under detection and pregnant woman known with male fetus.

2) Library Construction

Library was constructed according to plasma library construction requirements of Complete Genomics Inc.

3) Sequencing

The sequencing process was practiced strictly following the standard operating procedure of Complete Genomics Inc.

4) Data Analysing

Length distribution of DNA fragment was analyzed with reads obtained in the paired-end sequencing, and the process was shown in FIG. 1. Specific steps were as follows:

a) The length of DNA fragment in the peripheral blood sample obtained from each of 11 pregnant women under detection and 37 pregnant women known with male fetuses was calculated. Specifically, 19 bp at one end of each uniquely-mapped read and 12 bp at the other end of same uniquely-mapped read were choosen to determine start and end positions corresponding to the reference genome of the uniquely-mapped read, and thus the length of DNA fragment was obtained based on the start and end positions corresponding to the reference genome of the uniquely-ligned read.

b) For the peripheral blood sample obtained from each of 37 pregnant women known with male fetus, a plurality of window ranges (length range) were obtained through moving a window in a certain window length in accordance with a certain step size, a percentage of DNA moleculars present in each window range, i.e. a percentage of DNA moleculars present in each length range, was calucated. It should be noted that, the number of DNA moleculars present in each window range, i.e. distributed in each length range being divided by total number of DNA molecules was defined as the percentage of DNA molecular present in each window range. For example, 1 bp, 5 bp, 10 bp or 15 bp may be taken as the window and any size selected from 1 bp to the window length may be taken as the step size. Specifically, as an example, if 5 bp was taken as the window and 2 bp was taken as the step size, then distributions of DNA moleculars in [1 bp,5 bp], [2 bp,6 bp], [4 bp,8 bp], [6 bp,10 bp] and so on may be obtained. As another example, if 5 bp was taken as the window and 5 bp was taken as the step size, then distributions of DNA moleculars in [1 bp,5 bp], [6 bp,10 bp], [11 bp,15 bp] and so on may be obtained.

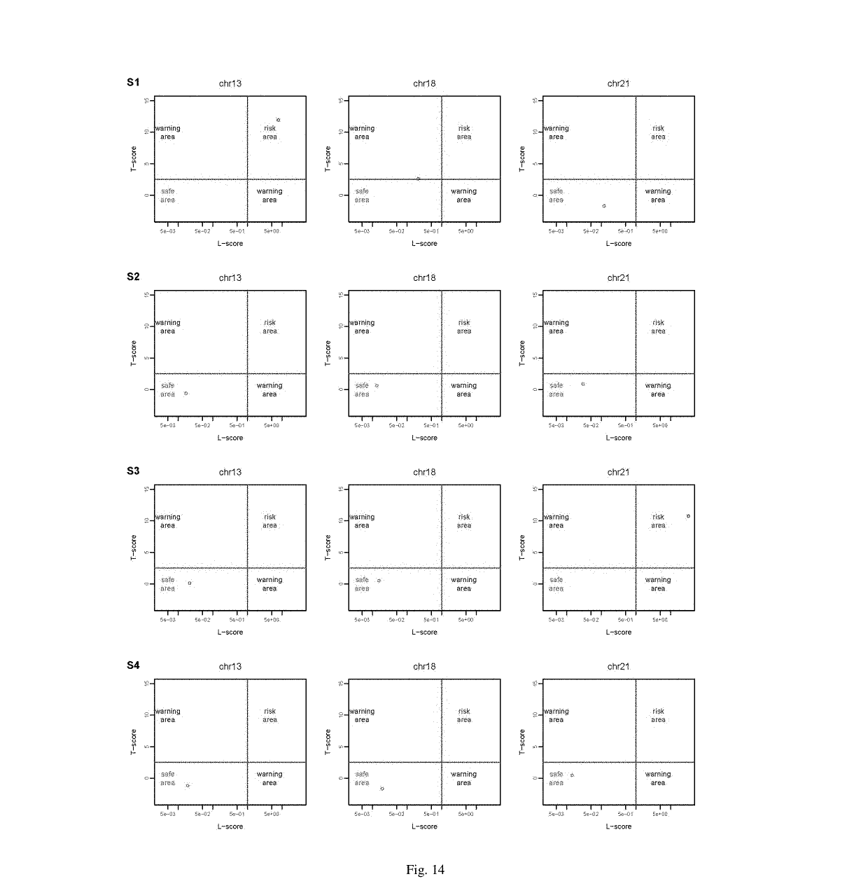

c) One or more ranges was/were selected, in which the percentages of DNA moleculars present for peripheral blood samples onbtained from 37 pregnant women known with male fetuses were strongly correlate to the known fetal fraction. For a certain length range, cell-free fetal DNA fractions in peripheral blood samples onbtained from 37 pregnant women known with male fetuses were estimated by chromosome Y (for the specific estimation method, reference may be made to Fuman Jiang, Jinghui Ren, et al. Noninvasive Fetal Trisomy (NIFTY) test: an advanced noninvasive prenatal diagnosis methodology for fetal autosomal and sex chromosomal aneuploidies. BMC Med Genomics. 2012 Dec. 1; 5:57. doi: 10.1186/1755-8794-5-57., the entire content of which is incorporated herein by reference) and a correlation coefficient between each cell-free fetal DNA fraction and percentage of DNA moleculars in a length falling into M was calculated. Further, a length range M with a maximum absolute value of correlation coefficient was selected, for example, M=185-204 bp, the correlation coefficient R=−0.87, as shown in FIG. 13; or M=121-150 bp, the correlation coefficient R=−0.6199.

d) Functional relationship between the percentage of DNA moleculars, in a peripheral blood sample obtained from a pregnant woman, present in the length range M and cell-free fetal DNA fraction (record as d) was determined. Specifically, with respect to the above 37 samples with known cell-free fetal DNA fractions d (samples obtained from 37 pregnant women known with male fetuses), a linear fitting graph was plotted using the percentages pᵢ (i=1, 2, . . . , 48) of DNA moleculars present in 185-204 bp and cell-free fetal DNA fractions dᵢ (i=1, 2, . . . , 48), such that a relationship formula therebetween was obtained: d=a*p+b, where d=0.0334*p+1.6657.

e) Length distribution of DNA fregment and the percentage of DNA moleculars present in M were obtained for each sample obtained from 11 pregnant women under detection. The percentage P of DNA fregment present in 185 bp˜0.204 bp was obtained for each sample obtained from pregnant women under detection, and results were shown in Table 1 below.

f) Cell-free fetal DNA fractions of samples under detection were estimated. Cell-free fetal DNA fraction dⱼ of each sample under detection was calculated directly according to the percentage pⱼ (j was a label of the sample under detection) of DNA fregment present in 185 bp˜204 bp obtained above for each sample under detection and the relationship formula d=a*p+b.

g) Estimated results of cell-free fetal DNA fractions of the samples under detection are shown in Table 1, in which for samples obtained from 37 pregnant women known with male fetuses, the cell-free fetal DNA fractions estimated by chrY are basically conformity with those obtained by the method of the present disclosure.

TABLE 1

| | | Cell-free fetal DNA | |
|---|---|---|---|
| | | fraction (estimated by | |
| | | the percentage of DNA | |
| Percentage of | Cell-free fetal | moleculars present in | |
| DNA moleculars | DNA fraction | 185 bp~204 bp, i.e. | |
| present in 185 | (estimated | the method of the | |
| bp~204 bp (%) | by chrY) | present disclosure) | Sample |
| 45.33932509 | 0.14872 | 0.145567224 | test sample |
| 45.85055949 | 0.12618 | 0.129861913 | test sample |
| 47.80808312 | 0.06752 | 0.06972606 | test sample |
| 46.78175523 | 0.10438 | 0.101255234 | test sample |
| 47.76095629 | 0.07148 | 0.071173814 | test sample |
| 45.18367884 | 0.15494 | 0.150348735 | test sample |
| 45.61282777 | 0.13413 | 0.13716512 | test sample |
| 48.50026322 | 0.04923 | 0.04846203 | test sample |
| 47.30076126 | 0.08555 | 0.085311176 | test sample |
| 47.12684798 | 0.09149 | 0.090653857 | test sample |
| 47.59287602 | 0.07892 | 0.076337302 | test sample |

### Example 2

Determination of sexuality of twins and the detection of the chromosome aneuploidy of twins were carried out using peripheral blood samples obtain from 11 pregnant women with twins described in Example 1, according to the method for determining sexuality of twins and the method for determining a chromosome aneuploidy of twins and besed on the results of cell-free fetal DNA fractions obtained in Example 1.

1. Determination of Sexuality of Fetus of Pregnant Woman Under Detection

Sexuality of each fetus of 11 pregnant women under detection was determined based on the results of cell-free fetal DNA fractions determined in Example 1 and according to the following steps:

a) fra.chry was calculated by chromosome Y;

b) fra.size was estimated by differences within a predetermined region between Mother and Fetal;

c) Determination standard:
I. If a value of fra.chry/fra.size is lower than 0.35, both fetuses of the twins are female;
II. If error! No reference source is found. If the value of fra.chry/fra.size is not lower than 0.35 and not greater than 0.7, the twins include a male fetus and a female fetus;
III. If error! No reference source is found. If the value of fra.chry/fra.size is greater than 0.7, both fetuses of the twins are male.

Results are shown in the table below:

Detection results of sexuality of fetus of 11 pregnant women under detection

| | | | fra.chry/ | Detection | Karyotype |
|---|---|---|---|---|---|
| Sample No. | fra.chry | fra.size | fra.size | result | results |
| S1 | 0.131431 | 0.118 | 1.113822 | [Male, | [Male, |
| | | | | Male] | Male] |
| S2 | −0.00014 | 0.126652 | −0.00114 | [Female, | [Female, |
| | | | | Female] | Female] |
| S3 | −0.00019 | 0.125 | −0.00151 | [Female, | [Female, |
| | | | | Female] | Female] |
| S4 | 0.141033 | 0.150125 | 0.939435 | [Male, | [Male, |
| | | | | Male] | Male] |
| S5 | 0.071202 | 0.088074 | 0.808433 | [Male, | [Male, |
| | | | | Male] | Male] |
| S6 | 0.06227 | 0.133 | 0.468195 | [Female, | [Female, |
| | | | | Male] | Male] |
| S7 | −0.00044 | 0.132 | −0.0033 | [Female, | [Female, |
| | | | | Female] | Female] |
| S8 | −0.00396 | 0.074 | −0.05353 | [Female, | [Female, |
| | | | | Female] | Female] |
| S9 | 0.185233 | 0.172 | 1.076936 | [Male, | [Male, |
| | | | | Male] | Male] |
| S10 | 0.072432 | 0.086 | 0.842233 | [Male, | [Male, |
| | | | | Male] | Male] |
| S11 | 0.000339 | 0.092792 | 0.003653 | [Female, | [Female, |
| | | | | Female] | Female] |

1. Determination of Chromosome Aneuploidy of Twins by Fetal Fractions

Chromosome aneuploidy of twins of 11 pregnant women under detection was determined by fetal fractions, based on the results of cell-free fetal DNA fractions determined in Example 1 and according to the following steps:

a) fra.chry was calculated by chromosome i (i=13,18,21);

b) fra.size was estimated by differences within a predetermined region between Mother and Fetal;

c) Determination standard:
I. If error! No reference source is found. If the value of fra.chry/fra.size is lower than 0.35, chromosome i in both fetuses of the twins are normal;
II. If error! No reference source is found. If the value of fra.chry/fra.size is not lower than 0.35 and not greater than 0.7, chromosome i in one fetus of the twins is trisome and in the other fetus of the twins is normal;
III. If error! No reference source is found. If the value of fra.chry/fra.size is greater than 0.7, chromosome i in both fetuses of the twins are trisome.

Results are shown in the table below:
Determination results of chromosome aneuploidy of twins of 11 pregnant women under detection by fetal fractions

| Sample | | | | | fra.chr13/ | fra.chr18/ | fra.chr21/ | Detection | Karyotype |
|---|---|---|---|---|---|---|---|---|---|
| No. | fra.chr13 | fra.chr18 | fra.chr21 | fra.size | fra.size | fra.size | fra.size | result | results |
| S1 | 0.113979 | 0.017835 | −0.01762 | 0.118 | 0.965921 | 0.151144 | −0.14933 | [T13,T13] | [T13,T13] |
| S2 | −0.00586 | 0.004833 | 0.009971 | 0.126652 | −0.0463 | 0.03816 | 0.078728 | [normal, | [normal,normal] |
| | | | | | | | | normal] | |
| S3 | 0.001131 | 0.003996 | 0.121607 | 0.125 | 0.009048 | 0.031968 | 0.972856 | [T21,T21] | [T21,T21] |
| S4 | −0.01175 | −0.01269 | 0.004841 | 0.150125 | −0.07829 | −0.08455 | 0.032246 | [normal, | [normal,normal] |
| | | | | | | | | normal] | |
| S5 | 2.20E−05 | −0.01032 | 0.011321 | 0.088074 | 0.00025 | −0.11721 | 0.128539 | [normal, | [normal,normal] |
| | | | | | | | | normal] | |
| S6 | 0.040963 | −0.00491 | −0.00723 | 0.133 | 0.307992 | −0.03693 | −0.05438 | [T13, | [T13,normal] |
| | | | | | | | | normal] | |
| S7 | 0.005557 | −0.01147 | 0.0754 | 0.132 | 0.042098 | −0.08688 | 0.571212 | [T21, | [T21,normal] |
| | | | | | | | | normal] | |
| S8 | 0.014679 | 0.031445 | −0.01884 | 0.074 | 0.198365 | 0.424932 | −0.25462 | [T18, | [T18, normal] |
| | | | | | | | | normal] | |
| S9 | 0.010402 | 0.082992 | 0.007482 | 0.172 | 0.060477 | 0.482512 | 0.0435 | [T18, | [T18,normal] |
| | | | | | | | | normal] | |
| S10 | 0.001905 | 0.074566 | −0.00051 | 0.086 | 0.022151 | 0.867052 | −0.00592 | [T18,T18] | [T18,T18] |
| S11 | 0.014591 | −0.00375 | −0.01728 | 0.092792 | 0.157244 | −0.04041 | −0.1862 | [normal, | [normal,normal] |
| | | | | | | | | normal] | |

2. Determination of Chromosome Aneuploidy of Twins of Pregnant Woman Under Detection by T Score & L Score

Chromosome aneuploidy of twins of 11 pregnant women under detection was determined by T score & L score, based on the results of cell-free fetal DNA fractions determined in Example 1 and according to the following steps:

a) Whole genome sequencing (WGS): the sample under detection was subjected to whole genome sequencing using the high-throughput platform Illumina.

b) Position information of effective reads was obtained: reads of a test sample were uniquely mapped to reference genome sequence hg19 to obtain position information, corresponding to the reference genome, of the uniquely-mapped reads.

c) Percentage of effective reads was obtained: Percentage of effective reads in each chromosome to total effective reads obtained in b) was obtained.

d) GC Correction:

UR and GC contents of each chromosome were fitted by using known data obtained from normal samples to obtain a relation formula: ERᵢ=fᵢ(GCᵢ)+εᵢ, and a mean value E̅R̅ᵢ of UR was calculated. For a sample to be analyzed, an ER value after correction was calculated according to the above relation formula and ER and GC of the sample.

E̅R̅ᵢⱼ=E̅R̅ᵢ+εᵢ=E̅R̅ᵢ+ERᵢⱼ−fᵢ(GCᵢⱼ)

e) T score was calculated: Tᵢ=(xᵢ−μᵢ)/σᵢ

where
i: a serial number of chromosome (i=13,18,21);
xᵢ: percentage of effective reads of the chromosome i in an analytic sample;
μᵢ: an average percentage of effective reads of the chromosome i selected as a reference system in a reference database;
σᵢ: a standard deviation of percentages of effective reads of chromosome i selected as the reference system in the reference database;

f) L score was calculated:

L score of chromosome i was determined according to the formula:

Lᵢ=log(d(Tᵢ,a))/log(d(T2ᵢ,a)),

where i represents the serial number of the chromosome, T2ᵢ=(xᵢ−μᵢ*(1+fra/2))/σᵢ; d(Tᵢ, a) and d(T2ᵢ, a) represent t distribution probability density function, a represents degree of freedom, fra represents the first cell-free fetal DNA fraction determined by the method in Example 1.

g) A four-quadrant diagram was ploted with T as vertical coordinate and L as horizontal coordinate by zoning with a first straight line where T=3 and a second straight line where L=0.8 (a sample with fetal frection<5% was determined to not meet the quality control), details were described as follows:
I. Both fetuses of the twins were determined to have trisome if a sample under detection was determined to be of the T score and the L score (T>3,L>0.8) falling into a first quadrant;
II. One fetus of the twins was determined to be of trisome and the other fetus of the twins was determined to be normal if a sample under detection is determined to be of the T score and the L score (T>3,L≦0.8) falling into a second quadrant;
III. Both fetuses of the twins were determined to be normal if a sample under detection is determined to be of the T score and the L score (T≦3,L≦0.8) falling into a third quadrant;
IV. The twins were determined to have a low fetal fraction if a sample under detection is determined to be of the T score and the L score (T≦3,L>0.8) falling into a fourth quadrant, such a sample did not meet the quality control.

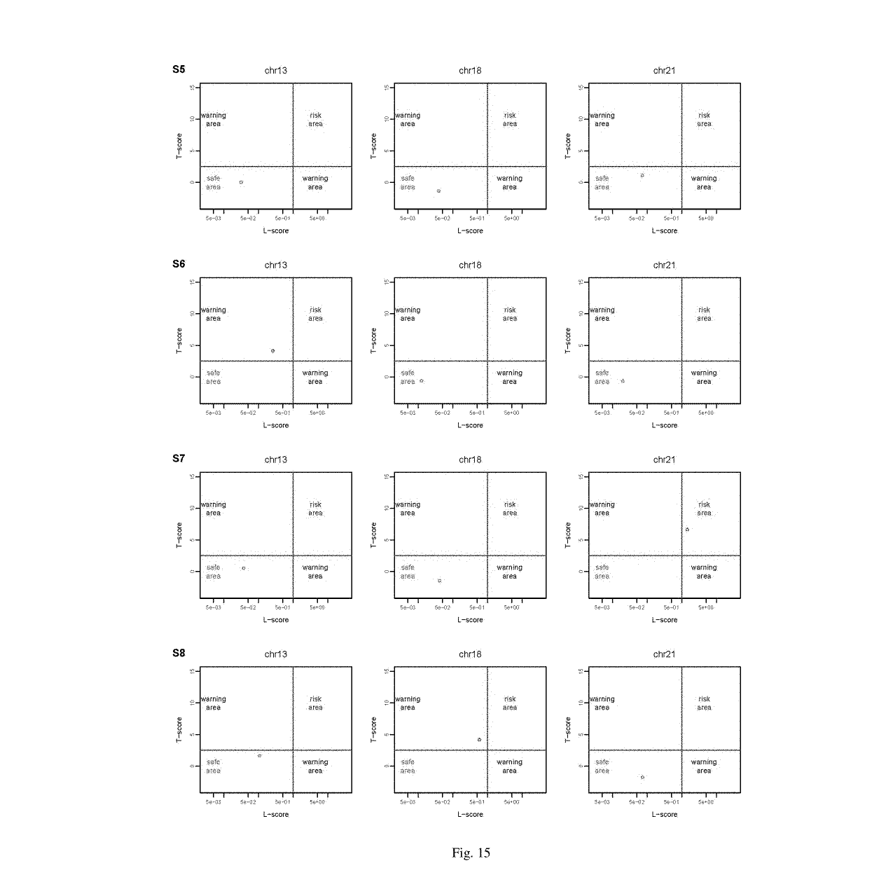

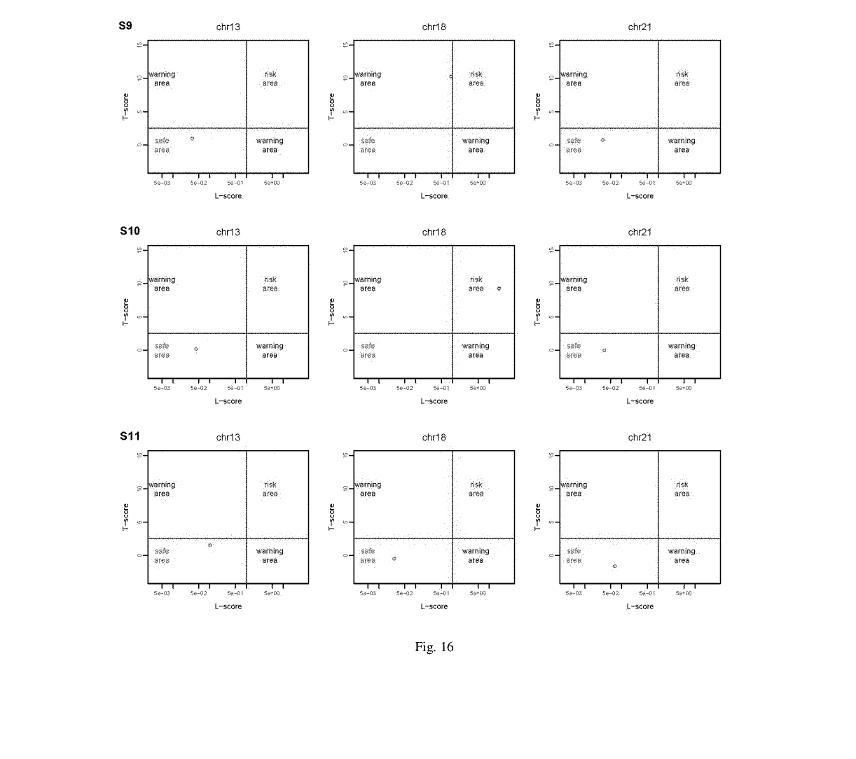

The four-quadrant diagrams ploted with T scores & L scores of 11 samples under detection are shown in FIGS. 14-16. It can be seen from FIGS. 14-16 that, the chromosome aneuploidy of twins of 11 pregnant women under detection determined by T score & L score is conformity with that determined in step 2 by fetal fraction.

### Example 3: Chimera Detection

In the following examples, a mixture of DNA fragments obtained from an abortion tissue and plasma obtained from a woman not pregnant in a certain proportion was simulated as a sample obtained from a pregnant woman. Chromosome number abnormality (trisome, complete monosome, trisome chimera, monosome chimera) of fetus (male) was detected according to the following method, which includes steps as follows.

1) Whole genome sequencing (WGS): the sample under detection was subjected to whole genome sequencing using the high-throughput platform.

2) Position information of effective reads was obtained: reads of a test sample were uniquely aligned to reference genome sequence to obtain position information, corresponding to the reference genome, of the uniquely-mapped reads.

3) Fraction of uniquely-mapped reads in each chromosome and percentage of the number of guanines (G bases) and Cytosines (C bases) of uniquely-mapped reads to total number of bases in each chromosome were obtained: it was obtained that the percentage of the number of effective reads in each chromosome in a sample under detection to the total number of effective reads thereof and the percentage of the number of G and C bases in effective reads in each chromosome to total number of bases thereof, using position information and base information of effective reads.

4) DNA fraction was calculated by chromosome i, which was maked as fra.chri

fra.chri=2*(chri.ER %/adjust.chri.ER %−1)*100%,

where fra.chri represents a cell-free fetal DNA fraction, i represents a serial number of a predetermined chromosome and i is any integer in the range of 1 to 22;

chri.ER %: ER % (short for effective reads rate, percentage of uniquely-mapped reads) of chromosome i in a sample;

adjust.chri.ER %: a theoretical value of ER % of chromosome i in a normal sample;

5) Fetal fraction was calculated by chromosome Y, which was maked as fra.chry;

fra.chry=(chry.ER %−Female.chr.ER %)/(Man.chry.UR %−Female.chry.ER %)*100%,

where chry.ER %:E_{R}% (short for effective reads rate, percentage of uniquely-mapped reads) of chromosome Y in a sample under detection;

Female.chry.ER %: an average of ER % of chromosome Y in a sample under detection obtained from a pregnant woman with a female fetus;

Man.chry.ER %: an average of ER % of chromosome Y in a sample under detection obtained from a man.

6) Determination standard:

I. If fra.chri/fra.chry<A1 (A1 is a certain constant, and A1>−1, such as −0.85), chromosome i of a fetus is a complete monosome;

II. fra.chri/fra.chryε[A₁, A₂] (A1 and A2 are certain constants, and −1<A1<A2<0, such as [−0.85,−0.3]), chromosome i of a fetus is a monosome chimera;

III. If fra.chri/fra.chryε[A₂, A₃] (A2 and A3 are certain constants, and A2<0<A3, such as [−0.3,0.3]), chromosome i of a fetus is normal;

IV. If fra.chri/fra.chryε[A₃, A₄] (A3 and A4 are certain constants, and 0<A3<A4<1, such as [0.3,0.85]),chromosome i of a fetus is a trisome chimera;

V. If fra.chri/fra.chry>A4 (A4 is a certain constant, such as 0.85), chromosome i of a fetus is a complete trisome.

3.1 Chromosome aneuploidy was detected to 19 samples (M1 . . . M19) each of which is a mixture of DNA fragments obtained from an abortion tissue and plasma obtained from a woman not pregnant (details was shown in table A) and 2 plasma samples (N1, N2) respectively obtained from 2 pregnant women with a male fetus (the male fetus in one pregnant woman was of T18 chimera, and the male fetus in the other pregnant woman was of T21 chimera).

1) Sample Collection and Treatment

2 ml peripheral blood was extracted for plasma separation.

2) Library Construction

Library was constructed according to plasma library construction requirements of Complete Genomics Inc.

3) Sequencing

The sequencing process was practiced strictly following the standard operating procedure of Complete Genomics Inc.

4) Data Analysing

a) Whole genome sequencing (WGS): the sample under detection was subjected to whole genome sequencing using the high-throughput platform (Lengthen of all cell-free DNA molecules were obtained by single-end sequencing or paired-end sequencing, which was important. The entire cell-free DNA molecule was required to be sequenced if by single-ended sequencing)

b) Position information of effective reads was obtained: reads of a test sample were uniquely aligned to reference genome sequence to obtain position information, corresponding to the reference genome, of the uniquely-mapped reads.

c) Percentage of effective reads was obtained: Percentage of effective reads in each chromosome to total effective reads obtained in b) was obtained.

d) GC Correction:

UR and GC contents of each chromosome were fitted by using known data obtained from normal samples to obtain a relation formula: ERᵢ=fᵢ(GCᵢ)+εᵢ, and a mean value E̅R̅ᵢ̅ of UR was calculated. For a sample to be analyzed, an ER value after correction was calculated according to the above relation formula and ER and GC of the sample.

E̅R̅ᵢⱼ=E̅R̅ᵢ+εᵢ=E̅R̅ᵢ+ERᵢⱼ−fᵢ(GC_{ijf})

e) fra.chri was calculated by chromosome i (i=13,18,21);

f) fra.chry was calculated by chromosome Y;

g) fra.size was calculated by the method for determining the cell-free fetal DNA fraction of the present disclosure;

h) T score was calculated: Tᵢ=(xᵢ−μᵢ)/σᵢ,

where i: a serial number of chromosome (i=1, 2 . . . 22);

xᵢ: a percentage of effective reads of the chromosome i in an analytic sample;

μᵢ: an average percentage of effective reads of the chromosome i selected as a reference system in a reference database;

σᵢ: a standard deviation of percentages of effective reads of chromosome i selected as the reference system in the reference database;

i) L score was calculated:

T2 value was firstly calculated: T2ᵢ=(xᵢ−μᵢ*(1+fra/2))/σᵢ,

L score was then calculated: Lᵢ=log(d(Tᵢ, a))/log(d(T2ᵢ,a)),

where d(Tᵢ, a) and d(T2ᵢ, a) represent t distribution probability density function, a represents degree of freedom, fra represents fra.chry or fra.size.
Chromosome aneuploidy of a sample under detection was determined by fra.chry which was estimated icy chromosome Y (as shown in table A).

a) Determination Standard:

I. If fra.chri/fra.chry<−0.85, chromosome i of a fetus is a complete monosome;

II. If fra.chri/fra.chryε[−0.85,−0.3], chromosome i of a fetus is a monosome chimera;

III. If fra.chri/fra.chryε[−0.3,0.3], chromosome i of a fetus is normal;

IV. If fra.chri/fra.chryε[0.3,0.85], chromosome i of a fetus is a trisome chimera;

V. If fra.chri/fra.chry>0.85, chromosome i of a fetus is a complete trisome.
Chromosome aneuploidy of a sample under detection was determined by T score & L score (fra.chry was estimated by chromosome Y) (as shown in table B)

a) A four-quadrant diagram was ploted based on T scores and L scores;

b) When T≦0, a four-quadrant diagram was ploted with T as vertical coordinate and absolute value of L as horizontal coordinate by zoning with a straight line where T=3 and a straight line where L=1 (a sample with fetal frection<5% was determined to not meet the quality control),

I. The fetus was determined to have monosome or monosome chimera if a sample under detection was determined to be of the T score and the L score (T>3,L>1) falling into a first quadrant;

II. The fetus was determined to have monosome chimera if a sample under detection is determined to be of the T score and the L score (T>3,L<1) falling into a second quadrant;

III. The fetus was determined to be normal if a sample under detection is determined to be of the T score and the L score (T≦3,L≦1) falling into a third quadrant;

IV. The fetus was determined to have a low fetal fraction if a sample under detection is determined to be of the T score and the L score (T≦3,L≧1) falling into a fourth quadrant, such a sample did not meet the quality control.

when T>0, a four-quadrant diagram was ploted with T as vertical coordinate and L as horizontal coordinate by zoning with a straight line where T=3 and a straight line where L=0.8 (a sample with fetal frection<5% was determined to not meet the quality control),

I. The fetus was determined to have trisome or trisome chimera if a sample under detection was determined to be of the T score and the L score (T>3,L>1) falling into a first quadrant;

II. The fetus was determined to have trisome chimera if a sample under detection is determined to be of the T score and the L score (T>3,L≦1) falling into a second quadrant;

III. The fetus was determined to be normal if a sample under detection is determined to be of the T score and the L score (T≦3,L≦1) falling into a third quadrant;

IV. The fetus was determined to have a low fetal fraction if a sample under detection is determined to be of the T score and the L score (T≦3,L>1) falling into a fourth quadrant, such a sample did not meet the quality control.
Chromosome aneuploidy of a sample under detection was determined by fra. size which was estimated by the method for determining the cell-free fetal DNA fraction of the present disclosure (as shown in table C)

b) Determination Standard:

I. If fra.chri/fra.chry<−0.85, chromosome i of a fetus is a complete monosome;

II. fra.chri/fra.chryε[−0.85,−0.3], chromosome i of a fetus is a monosome chimera;

III. fra.chri/fra.chryε[−0.3,0.3], chromosome i of a fetus is normal;

IV. If fra.chri/fra.chryε[0.3,0.85], chromosome i of a fetus is a trisome chimera;

V. If fra.chri/fra.chry>0.85, chromosome i of a fetus is a complete trisome
Chromosome aneuploidy of a sample under detection was determined by T score & L score (fra. size was estimated by the method for determining the cell-free fetal DNA fraction of the present disclosure) (as shown in table D, FIG. 2)

a) A four-quadrant diagram was plotted based on T scores and L scores;

b) When T≦0, a four-quadrant diagram was plotted with T as vertical coordinate and absolute value of L as horizontal coordinate by zoning with a straight line where T=3 and a straight line where L=1 (a sample with fetal frection<5% was determined to not meet the quality control),

I. The fetus was determined to have monosome or monosome chimera if a sample under detection was determined to be of the T score and the L score (T>3,L>1) falling into a first quadrant;

II. The fetus was determined to have monosome chimera if a sample under detection is determined to be of the T score and the L score (T>3,L≦1) falling into a second quadrant;

III. The fetus was determined to be normal if a sample under detection is determined to be of the T score and the L score (T≦3,L≦1) falling into a third quadrant;

IV. The fetus was determined to have a low fetal fraction if a sample under detection is determined to be of the T score and the L score (T≦3,L>1) falling into a fourth quadrant, such a sample did not meet the quality control;

When T>0, a four-quadrant diagram was ploted with T as vertical coordinate and Las horizontal coordinate by zoning with a straight line where T=3 and a straight line where L=1 (a sample with fetal frection <5% was determined to not meet the quality control),

I. The fetus was determined to have trisome or trisome chimera if a sample under detection was determined to be of the T score and the L score (T>3,L>1) falling into a first quadrant;

II. The fetus was determined to have trisome chimera if a sample under detection is determined to be of the T score and the L score (T>3,L≦1) falling into a second quadrant;

III. The fetus was determined to be normal if a sample under detection is determined to be of the T score and the L score (T≦3,L<1) falling into a third quadrant;

IV. The fetus was determined to have a low fetal fraction if a sample under detection is determined to be of the T score and the L score (T≦3,L>1) falling into a fourth quadrant, such a sample did not meet the quality control.

In this embodiment, a negative sample used herein was a plasma sample obtained from a normal woman who was not pregnant; a positive sample was prepared by mixing DNA fragments which were obtained by randomly breaking DNAs from an abortion tissue in accordance with a size ranging from 150 bp to 200 bp and plasma obtained from a normal woman who was not pregnant; (T21, T18 each represents male fetus; T13 represents female fetus); a positive chimeric sample was prepared by mixing placental tissue DNA fragments (which were obtained by randomly breaking DNAs from an placental tissue in accordance with a size ranging from 150 bp to 200 bp), Chinese cell line DNA fragments (which were obtained by randomly breaking DNAs from Chinese cell line in accordance with a size ranging from 150 bp to 200 bp) and plasma obtained from a normal woman; (T21, T18 each represents male fetus; T13 represents female fetus).

| | Abortion tissue | Fetal | Chimeric | Expression |
|---|---|---|---|---|
| Sample No. | karyotype | fraction | ratio | form |
| M1 | trisome 13 | 3.5% | 0 | T13-3.5% |
| M2 | trisome 13 | 5% | 0 | T13-5% |
| M3 | trisome 13 | 8% | 0 | T13-8% |
| M4 | trisome 13 | 8% | 0 | T13-8% |
| M5 | trisome 18 | 10% | 30% | T18-10%-30% |
| M6 | trisome 18 | 10% | 70% | T18-10%-70% |
| M7 | trisome 18 | 10% | 70% | T18-10%-70% |
| M8 | trisome 18 | 10% | 70% | T18-10%-70% |
| M9 | trisome 18 | 3.5% | 0 | T18-3.5% |
| M10 | trisome 18 | 5% | 0 | T18-5% |
| M11 | trisome 18 | 5% | 0 | T18-5% |
| M12 | trisome 18 | 8% | 0 | T18-8% |
| M13 | trisome 18 | 10% | 30% | T21-10%-30% |
| M14 | trisome 18 | 10%- | 70% | T21-10%-70% |
| M15 | trisome 18 | 10% | 70% | T21-10%-70% |
| M16 | trisome 18 | 10% | 70% | T21-10%-70% |
| M17 | trisome 18 | 3.5% | 0 | T21-3.5% |
| M18 | trisome 18 | 5% | 0 | T21-5% |
| M19 | trisome 18 | 8% | 0 | T21-8% |

| Sample No. | Karyotype results |
|---|---|
| N1 | 47, XN + 18[3]/46, XN[20] |
| N2 | 47, XN + 21[30]/46, XN[18] |

TABLE A
Chromosome aneuploidy detection by mixed DNA fraction estimated by chromosome Y

| Sample | | | | | fra.chr13/ | fra.chr18/ | fra.chr21/ | |
|---|---|---|---|---|---|---|---|---|
| No. | fra.chr13 | fra.chr18 | fra.chr21 | fra.chry | fra.chry | fra.chry | fra.chry | Detection result |
| M1 | 0.03531 | −4.8E−06 | −0.00742 | 0.00069 | | | | T13-3.5% |
| M2 | 0.04731 | −0.01207 | 0.007764 | −0.00002 | | | | T13-5% |
| M3 | 0.08136 | −0.00477 | −0.00129 | −0.0002 | | | | T13-8% |
| M4 | 0.0796 | −0.00738 | −0.0013 | −0.00157 | | | | T13-8% |
| M5 | −0.01079 | 0.03401 | −0.00442 | 0.105215 | −0.10259 | 0.323243 | −0.04197 | T18-10%-30% |
| M6 | −0.00192 | 0.069573 | −0.00043 | 0.090435 | −0.02127 | 0.769319 | −0.00471 | T18-10%-70% |
| M7 | 0.001756 | 0.058363 | −0.00968 | 0.087785 | 0.020009 | 0.664844 | −0.11022 | T18-10%-70% |
| M8 | −0.01081 | 0.082063 | −0.00917 | 0.106115 | −0.1019 | 0.773343 | −0.08638 | T18-10%-70% |
| M9 | 0.007816 | 0.0341 | −0.00038 | 0.03723 | 0.209951 | 0.915928 | −0.01009 | T18-3.5% |
| M10 | 0.013116 | 0.0512 | −0.00607 | 0.04843 | 0.270834 | 1.057196 | −0.12525 | T18-5% |
| M11 | 0.000956 | 0.0513 | −0.01176 | 0.0566 | 0.016899 | 0.90636 | −0.2077 | T18-5% |
| M12 | −0.00237 | 0.07693 | −0.004 | 0.08877 | −0.02674 | 0.866622 | −0.04501 | T18-8% |
| M13 | 0.001246 | −0.01028 | 0.04323 | 0.110455 | 0.011285 | −0.09311 | 0.391381 | T21-10%-30% |
| M14 | 0.000266 | −0.01586 | 0.06554 | 0.092805 | 0.002871 | −0.17095 | 0.706212 | T21-10%-70% |
| M15 | −0.00041 | 0.004795 | 0.07461 | 0.110035 | −0.00376 | 0.043578 | 0.678057 | T21-10%-70% |
| M16 | −0.00235 | −0.00759 | 0.06985 | 0.097155 | −0.02422 | −0.07817 | 0.718954 | T21-10%-70% |
| M17 | 0.005776 | 0.010345 | 0.0481 | 0.03478 | 0.166086 | 0.297445 | 1.382979 | T21-3.5% |
| M18 | 0.007786 | 0.006345 | 0.0557 | 0.04853 | 0.160447 | 0.130747 | 1.147744 | T21-5% |
| M19 | 0.009846 | −0.00828 | 0.07449 | 0.06855 | 0.143639 | −0.12086 | 1.086652 | T21-8% |
| N1 | 0.007053 | 0.043212 | 0.000938 | 0.110337 | 0.063922 | 0.391637 | 0.008501 | 47,XN |
| | | | | | | | | +18[3]/46,XN[20] |
| N2 | −0.01105 | 0.002921 | 0.081665 | 0.167834 | −0.06581 | 0.017404 | 0.486582 | 47,XN |
| | | | | | | | | +21[30]/46,XN[18] |

Note:
fra.chr13: represents a mixed DNA fraction estimated by chromosome 13;
fra.chr18: represents a mixed DNA fraction estimated by chromosome 18;
fra.chr21: represents a mixed DNA fraction estimated by chromosome 21;
fra.chry: represents a mixed DNA fraction calculated by chromosome Y;
T21-10%-30%: DNA fragments obtained from cell line with trisome 21 being mixed with woman plasma in accordance with a fraction of 10% and a chimeric ratio of 30%.

TABLE B
Chromosome aneuploidy detection by T score & L score (mixed DNA fraction was estimated by
chromosome Y)

| Sample | | | | | | | | Detection |
|---|---|---|---|---|---|---|---|---|
| No. | T.chr13 | L.chr13 | T.chr18 | L.chr18 | T.chr21 | L.chr21 | fra.chry | result |
| M1 | 4.345751 | | −0.00053 | | −0.60057 | | 0.00069 | T13-3.5% |
| M2 | 5.436515 | | −1.216 | | 0.52145 | | −0.00002 | T13-5% |
| M3 | 10.46607 | | −0.47762 | | −0.09072 | | −0.0002 | T13-8% |
| M4 | 9.126057 | | −0.70384 | | −0.08565 | | −0.00157 | T13-8% |
| M5 | −1.43378 | 0.018171 | 4.16099 | 0.292408 | −0.39025 | 0.023403 | 0.105215 | T18-10%-30% |
| M6 | −0.20845 | 0.015392 | 7.977908 | 12.56388 | −0.03313 | 0.031699 | 0.090435 | T18-10%-70% |
| M7 | 0.210445 | 0.015766 | 7.151443 | 4.550099 | −0.82976 | 0.035673 | 0.087785 | T18-10%-70% |
| M8 | −1.19968 | 0.019721 | 8.763087 | 10.86877 | −0.7035 | 0.030472 | 0.106115 | T18-10%-70% |
| M9 | 0.963351 | 0.189495 | 3.96625 | 9.33211 | −0.03142 | 0.162273 | 0.03723 | T18-3.5% |
| M10 | 1.8534 | 0.251063 | 6.429505 | 19.56264 | −0.51602 | 0.082144 | 0.04843 | T18-5% |
| M11 | 0.112697 | 0.031697 | 6.659763 | 23.63495 | −1.01942 | 0.072816 | 0.0566 | T18-5% |
| M12 | −0.24379 | 0.016926 | 8.332999 | 25.4093 | −0.2922 | 0.035987 | 0.08877 | T18-8% |
| M13 | 0.164157 | 0.010591 | −1.22048 | 0.017938 | 3.76578 | 0.491369 | 0.110455 | T21-10%-30% |
| M14 | 0.037473 | 0.013915 | −1.90428 | 0.036927 | 5.667693 | 4.554072 | 0.092805 | T21-10%-70% |
| M15 | −0.05039 | 0.011486 | 0.536781 | 0.017359 | 5.897363 | 3.910694 | 0.110035 | T21-10%-70% |
| M16 | −0.28883 | 0.01242 | −0.86821 | 0.018234 | 6.167815 | 5.406755 | 0.097155 | T21-10%-70% |
| M17 | 0.581767 | 0.19567 | 1.077291 | 0.427888 | 3.582341 | 4.827084 | 0.03478 | T21-3.5% |
| M18 | 0.93815 | 0.145201 | 0.654071 | 0.129238 | 3.958377 | 8.227322 | 0.04853 | T21-5% |
| M19 | 1.201005 | 0.051788 | −1.03581 | 0.032569 | 6.795784 | 21.00502 | 0.06855 | T21-8% |
| N1 | 0.703062 | 0.022374 | 5.418144 | 0.435182 | 0.081371 | 0.02066 | 0.110337 | 47,XN |
| | | | | | | | | +18[3]/46,XN[20] |
| N2 | −0.97092 | 0.012246 | 0.355485 | 0.005614 | 7.170205 | 0.903486 | 0.167834 | 47,XN |
| | | | | | | | | 21[30]/46,XN[18] |

Note:
T.chr13: T score of chromosome 13;
L.chr13: L score of chromosome 13;
T.chr18: T score of chromosome 18;
L.chr18: L score of chromosome 18;
T.chr21: T score of chromosome 21;
L.chr21: L score of chromosome 21;
fra.chry: mixed fetal DNA fraction estimated by chromosome Y;
T21-10%-30%: DNA fragments obtained from cell line with trisome 21 being mixed with woman plasma in accordance with a fraction of 10% and a chimeric ratio of 30%.

TABLE C
Chromosome aneuploidy detection by fra.size

| Sample | | | | | fra.chr13/ | fra.chr18/ | fra.chr21/ | |
|---|---|---|---|---|---|---|---|---|
| No. | fra.chr13 | fra.chr18 | fra.chr21 | fra.size | fra.size | fra.size | fra.size | Detection result |
| M1 | 0.03531 | −4.8E−06 | −0.00742 | 0.0369 | 0.956911 | −0.00013 | −0.20097 | T13-3.5% |
| M2 | 0.04731 | −0.01207 | 0.007764 | 0.05519 | 0.857221 | −0.21879 | 0.140682 | T13-5% |
| M3 | 0.08136 | −0.00477 | −0.00129 | 0.08462 | 0.961475 | −0.05643 | −0.01519 | T13-8% |
| M4 | 0.0796 | −0.00738 | −0.0013 | 0.07332 | 1.085652 | −0.10072 | −0.01767 | T13-8% |
| M5 | −0.01079 | 0.03401 | −0.00442 | 0.1071 | −0.10078 | 0.317554 | −0.04123 | T18-10%-30% |
| M6 | −0.00192 | 0.069573 | −0.00043 | 0.0928 | −0.02073 | 0.749713 | −0.00459 | T18-10%-70% |
| M7 | 0.001756 | 0.058363 | −0.00968 | 0.1058 | 0.016602 | 0.551638 | −0.09145 | T18-10%-70% |
| M8 | −0.01081 | 0.082063 | −0.00917 | 0.1233 | −0.0877 | 0.665558 | −0.07434 | T18-10%-70% |
| M9 | 0.007816 | 0.0341 | −0.00038 | 0.0432 | 0.180937 | 0.789352 | −0.0087 | T18-3.5% |
| M10 | 0.013116 | 0.0512 | −0.00607 | 0.05688 | 0.230599 | 0.900141 | −0.10664 | T18-5% |
| M11 | 0.000956 | 0.0513 | −0.01176 | 0.0438 | 0.021838 | 1.171233 | −0.2684 | T18-5% |
| M12 | −0.00237 | 0.07693 | −0.004 | 0.06726 | −0.03529 | 1.14377 | −0.05941 | T18-8% |
| M13 | 0.001246 | −0.01028 | 0.04323 | 0.0705 | 0.017681 | −0.14588 | 0.613191 | T21-10%-30% |
| M14 | 0.000266 | −0.01586 | 0.06554 | 0.1056 | 0.002524 | −0.15024 | 0.620644 | T21-10%-70% |
| M15 | −0.00041 | 0.004795 | 0.07461 | 0.1205 | −0.00343 | 0.039794 | 0.61917 | T21-10%-70% |
| M16 | −0.00235 | −0.00759 | 0.06985 | 0.1165 | −0.0202 | −0.06519 | 0.599571 | T21-10%-70% |
| M17 | 0.005776 | 0.010345 | 0.0481 | 0.0409 | 0.141234 | 0.252938 | 1.176039 | T21-3.5% |
| M18 | 0.007786 | 0.006345 | 0.0557 | 0.04543 | 0.171395 | 0.139669 | 1.226062 | T21-5% |
| M19 | 0.009846 | −0.00828 | 0.07449 | 0.07848 | 0.125465 | −0.10557 | 0.949159 | T21-8% |
| N1 | 0.007053 | 0.043212 | 0.000938 | 0.102547 | 0.068778 | 0.421387 | 0.009147 | 47,XN |
| | | | | | | | | +18[3]/46,XN[20] |
| N2 | −0.01105 | 0.002921 | 0.081665 | 0.198228 | −0.05572 | 0.014736 | 0.411975 | 47,XN |
| | | | | | | | | +21[30]/46,XN[18] |

Note:
fra.chr13: represents a mixed DNA fraction estimated by chromosome 13;
fra.chr18: represents a mixed DNA fraction estimated by chromosome 18;
fra.chr21: represents a mixed DNA fraction estimated by chromosome 21;
fra.size: represents a mixed DNA fraction estimated by the method of the present disclosure for determining a fraction of cell-free nucleic acids from a predetermined source in a biological sample;
T21-10%-30%: DNA fragments obtained from cell line with trisome 21 being mixed with woman plasma in accordance with a fraction of 10% and a chimeric ratio of 30%.

TABLE D
Chromosome aneuploidy detection by T score & L score (fra.size)

| Sample | | | | | | | | |
|---|---|---|---|---|---|---|---|---|
| No. | T.chr13 | L.chr13 | T.chr18 | L.chr18 | T.chr21 | L.21 | fra.size | Detection result |
| M1 | 4.345751 | 10.86269 | −0.00053 | 0.06264 | −0.60057 | 0.110064 | 0.0369 | T13-3.5% |
| M2 | 5.436515 | 13.40864 | −1.216 | 0.040421 | 0.52145 | 0.080194 | 0.05519 | T13-5% |
| M3 | 10.46607 | 36.3034 | −0.47762 | 0.013724 | −0.09072 | 0.026097 | 0.08462 | T13-8% |
| M4 | 9.126057 | 29.68988 | −0.70384 | 0.019292 | −0.08565 | 0.033903 | 0.07332 | T13-8% |
| M5 | −1.43378 | 0.015183 | 4.16099 | 0.216731 | −0.39025 | 0.019427 | 0.1071 | T18-10%-30% |
| M6 | −0.20845 | 0.01467 | 7.977908 | 10.52499 | −0.03313 | 0.030232 | 0.0928 | T18-10%-70% |
| M7 | 0.210445 | 0.011014 | 7.151443 | 1.813942 | 0.026168 | −0.82976 | 0.1058 | T18-10%-70% |
| M8 | −1.19968 | 0.015259 | 8.763087 | 4.176647 | −0.7035 | 0.023665 | 0.1233 | T18-10%-70% |
| M9 | 0.963351 | 0.136353 | 3.96625 | 6.810597 | −0.03142 | 0.126392 | 0.0432 | T18-3.5% |
| M10 | 1.8534 | 0.166895 | 6.429505 | 20.92116 | −0.51602 | 0.062989 | 0.05688 | T18-5% |
| M11 | 0.112697 | 0.051911 | 6.659763 | 13.06329 | −1.01942 | 0.107019 | 0.0438 | T18-5% |
| M12 | −0.24379 | 0.028322 | 8.332999 | 19.64862 | −0.2922 | 0.058661 | 0.06726 | T18-8% |
| M13 | 0.164157 | 0.025146 | −1.22048 | 0.037995 | 3.76578 | 2.372928 | 0.0705 | T21-10%-30% |
| M14 | 0.037473 | 0.010899 | −1.90428 | 0.030091 | 5.667693 | 2.443191 | 0.1056 | T21-10%-70% |
| M15 | −0.05039 | 0.009693 | 0.536781 | 0.014533 | 5.897363 | 2.522845 | 0.1205 | T21-10%-70% |
| M16 | −0.28883 | 0.008906 | −0.86821 | 0.013317 | 6.167815 | 2.195736 | 0.1165 | T21-10%-70% |
| N1 | 0.703062 | 0.030365 | 5.418144 | 0.576578 | 0.081371 | 0.02724 | 0.102547 | 47,XN |
| | | | | | | | | +18[3]/46,XN[20] |
| N2 | −0.97092 | 0.013866 | 0.355485 | 0.007248 | 7.170205 | 0.553012 | 0.198228 | 47,XN |
| | | | | | | | | +21[30]/46,XN[18] |

Note:
T.chr13: T score of chromosome 13;
L.chr13: L score of chromosome 13;
T.chr18: T score of chromosome 18;
L.chr18: L score of chromosome 18;
T.chr21: T score of chromosome 21;
L.chr21: L score of chromosome 21;
fra.size: mixed DNA fraction estimated by estimated by the method of the present disclosure for determining a fraction of cell-free nucleic acids from a predetermined source in a biological sample;
T21-10%-30%: DNA fragments obtained from cell line with trisome 21 being mixed with woman plasma in accordance with a fraction of 10% and a chimeric ratio of 30%.

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example,” “in an example,” “in a specific example,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure, and the scope of the present disclosure is defined by the claims and its equivalents.

1. A method for determining a fraction of cell-free nucleic acids from a predetermined source in a biological sample, comprising:
performing sequencing on cell-free nucleic acids contained in the biological sample, so as to obtain a sequencing result consisting of a plurality of sequencing data;
determining the number of the cell-free nucleic acids in a length falling into a predetermined range in the biological sample based on the sequencing result; and
determining the fraction of the cell-free nucleic acids from the predetermined source in the biological sample based on the number of the cell-free nucleic acids in the length falling into the predetermined range.

2. The method according to claim 1, wherein the biological sample is a peripheral blood sample.

3. The method according to claim 2, wherein the cell-free nucleic acid from the predetermined source is selected from one of the followings:
cell-free fetal nucleic acids or cell-free maternal nucleic acids in a peripheral blood sample obtained from a pregnant woman, or
cell-free tumor derived nucleic acids or cell-free non-tumor derived nucleic acids in a peripheral blood sample obtained from a subject suffering from tumor, suspected to suffer from tumor or subjected to tumor screening.

4-5. (canceled)

6. The method according to of claim 1, wherein determining the number of the cell-free nucleic acids in the length falling into the predetermined range in the biological sample based on the sequencing result further comprises:
aligning the sequencing result to a reference genome, so as to construct a dataset consisting of a plurality of uniquely-mapped reads, where each read in the dataset can be mapped to a position of the reference genome only;
determining a length of the cell-free nucleic acid corresponding to each uniquely-mapped read in the dataset; and
determining the number of the cell-free nucleic acids in the length falling into the predetermined range.

7. The method according to claim 6, wherein determining the length of the cell-free nucleic acid corresponding to each uniquely-mapped read in the dataset further comprises:
determining the length of each read uniquely mapped to the reference genome as the length of the cell-free nucleic acid corresponding to the read.

8. The method according to claim 6, wherein in the case that the cell-free nucleic acids in the biological sample are sequenced by the paired-end sequencing, determining the length of the cell-free nucleic acid corresponding to each uniquely-mapped read in the dataset further comprises:
determining a position, corresponding to the reference genome, of 5′-end of the cell-free nucleic acid, based on sequencing data at one end of each uniquely-mapped read obtained in the paired-end sequencing;
determining a position, corresponding to the reference genome, of 3′-end of the cell-free nucleic acid, based on sequencing data at the other end of same uniquely-mapped read obtained in the paired-end sequencing; and
determining the length of the cell-free nucleic acid based on the position of 5′-end of the cell-free nucleic acid and the position of 3′-end of the cell-free nucleic acid.

9. The method according to claim 1, wherein the predetermined range is determined based on a plurality of control samples, in each of which the fraction of the cell-free nucleic acids from the predetermined source is known.

10. (canceled)

11. The method according to claim 9, wherein the predetermined range is determined by the following steps:
(a) determining lengths of the cell-free nucleic acids in the plurality of control samples;
(b) setting a plurality of candidate length ranges, and determining a percentage of the cell-free nucleic acids, obtained from each of the plurality of control samples, present in each candidate length range;
(c) determining a correlation coefficient between each candidate length range and the fraction of the cell-free nucleic acids from the predetermined source, based on the percentage of the cell-free nucleic acids, obtained from each of the plurality of control samples, present in each candidate length range and the fraction of the cell-free nucleic acids from the predetermined source in the control samples; and
(d) determining at least one candidate length range or a combination of the candidate length ranges as the predetermined range based on the correlation coefficient.

12-13. (canceled)

14. The method according to claim 9, wherein determining the fraction of the cell-free nucleic acids from the predetermined source in the biological sample based on the number of the cell-free nucleic acids in the length falling into the predetermined range further comprises:
determining a percentage of the cell-free nucleic acids present in the predetermined range based on the number of cell-free nucleic acids in the length falling into the predetermined range; and
determining the fraction of the cell-free nucleic acids from the predetermined source in the biological sample, based on the percentage of the cell-free nucleic acids present in the predetermined range, according to a predetermined function,
wherein the predetermined function is determined based on the plurality of control samples.

15. The method according to claim 14, wherein the predetermined function is obtained by following steps:
(i) determining the percentage of the cell-free nucleic acids, obtained from each control sample, present in the predetermined range; and
(ii) fitting the percentage of the cell-free nucleic acids, obtained from each control sample, present in the predetermined range with the known fraction of the cell-free nucleic acid from the predetermined source, to determine the predetermined function.

16. The method according to claim 15, wherein the percentage of the cell-free nucleic acids, obtained from each control sample, present in the predetermined range is fitted with the known fraction of the cell-free nucleic acid from the predetermined source by a linear fitting.

17. The method according to claim 1, wherein the cell-free nucleic acid from the predetermined source is cell-free fetal nucleic acid obtained from a peripheral blood sample of a pregnant woman, and the predetermined range is 185 bp to 204 bp.

18. The method according to claim 9, wherein the control sample is a peripheral blood sample obtained from a pregnant woman in which the fraction of the cell-free fetal nucleic acids is known.

19. The method according to claim 18, wherein the control sample is a peripheral blood sample obtained from a pregnant woman with a normal male fetus, in which the fraction of the cell-free fetal nucleic acids is known to be determined by chromosome Y.

20-37. (canceled)

38. A method for determining sexuality of twins, comprising:
performing sequencing on cell-free nucleic acids contained in a peripheral blood sample obtained from a pregnant woman with twins, so as to obtain a sequencing result consisting of a plurality of sequencing data;
determining a first cell-free fetal DNA fraction based on the sequencing data, by the method according to claim 1;
determining a second cell-free fetal DNA fraction based on a sequencing data derived from chromosome Y in the sequencing result; and
determining the sexuality of the twins based on the first cell-free fetal DNA fraction and the second cell-free fetal DNA fraction.

39. The method according to claim 38, wherein the second cell-free fetal DNA fraction is determined according to the following formula:
fra.chry=(chry.ER %−Female.chry.ER %)/(Man.chry.ER %−Female.chry.ER %)*100%,
where fra.chry represents the second cell-free fetal DNA fraction, chry.ER % represents a percentage of the sequencing data derived from chromosome Y in the sequencing result to total sequencing data;
Female.chry.ER % represents an average percentage of sequencing data of cell-free nucleic acids derived from chromosome Y in a peripheral blood sample obtained from a pregnant woman predetermined to be with a normal female fetus to total sequencing data thereof; and
Man.chry.ER % represents an average percentage of sequencing data of cell-free nucleic acids derived from chromosome Y in a peripheral blood sample obtained from a healthy man to total sequencing data thereof.

40. The method according to claim 38, wherein determining the sexuality of the twins based on the first cell-free fetal DNA fraction and the second cell-free fetal DNA fraction further comprises:
(a) determining a ratio of the second cell-free fetal DNA fraction to the first cell-free fetal DNA fraction; and
(b) determining the sexuality of the twins by comparing the ratio determined in (a) with a first threshold and a second threshold predetermined.

41. The method according to claim 40, wherein the first threshold is determined based on a plurality of control samples obtained from pregnant women known with female twins, and the second threshold is determined based on a plurality of control samples obtained from pregnant women known with male twins.

42. The method according to claim 41, wherein
both fetuses of the twins are female if the ratio of the second cell-free fetal DNA fraction to the first cell-free fetal DNA fraction is lower than the first threshold,
both fetuses of the twins are male if the ratio of the second cell-free fetal DNA fraction to the first cell-free fetal DNA fraction is greater than the second threshold, and
the twins include a male fetus and a female fetus if the ratio of the second cell-free fetal DNA fraction to the first cell-free fetal DNA fraction is equal to the first threshold or the second threshold, or between the first threshold and the second threshold.

43. The method according to claim 42, wherein the first threshold is 0.35 and the second threshold is 0.7.

44-49. (canceled)

50. A method for detecting a chromosome aneuploidy of twins, comprising:
performing sequencing on cell-free nucleic acids contained in a peripheral blood sample obtained from a pregnant woman with twins, so as to obtain a sequencing result consisting of a plurality of sequencing data;
determining a first cell-free fetal DNA fraction, based on the sequencing data, by the method according to claim 1;
determining a third cell-free fetal DNA fraction, based on a sequencing data derived from a predetermined chromosome in the sequencing result; and
determining whether the twins under detection have aneuploidy with respect to the predetermined chromosome based on the first cell-free fetal DNA fraction and the third cell-free fetal DNA fraction.

51. The method according to claim 50, wherein the third cell-free fetal DNA fraction is determined according to the following formula:
fra.chri=2*(chri.ER %/adjust.chri.ER %−1)*100%,
where fra.chri represents the third cell-free fetal DNA fraction, i represents a serial number of the predetermined chromosome, and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with normal twins to total sequencing data thereof.

52. The method according to claim 51, wherein determining whether the twins under detection have aneuploidy with respect to the predetermined chromosome based on the first cell-free fetal DNA fraction and the third cell-free fetal DNA fraction further comprises:
(a) determining a ratio of the third cell-free fetal DNA fraction to the first cell-free fetal DNA fraction; and
(b) determining whether the twins under detection have aneuploidy with respect to the predetermined chromosome by comparing the ratio determined in (a) with a third threshold and a fourth threshold predetermined.

53. The method according to claim 52, wherein the third threshold is determined based on a plurality of control samples obtained from pregnant women with twins known not to have aneuploidy with respect to the predetermined chromosome, and the fourth threshold is determined based on a plurality of control samples obtained from pregnant women with twins known to have aneuploidy with respect to the predetermined chromosome.

54. The method according to claim 53, wherein
both fetuses of the twins have no aneuploidy with respect to the predetermined chromosome if the ratio of the third cell-free fetal DNA fraction to the first cell-free fetal DNA fraction is lower than the third threshold,
both fetuses of the twins have aneuploidy with respect to the predetermined chromosome if the ratio of the third cell-free fetal DNA fraction to the first cell-free fetal DNA fraction is greater than the fourth threshold, and
one fetus of the twins has the aneuploidy with respect to the predetermined chromosome, while the other fetus of the twins has no aneuploidy with respect to the predetermined chromosome if the ratio of the third cell-free fetal DNA fraction to the first cell-free fetal DNA fraction is equal to the third threshold or the fourth threshold, or between the third threshold and the fourth threshold.

55. The method according to claim 54, wherein the third threshold is 0.35 and the fourth threshold is 0.7.

56. The method according to claim 50, wherein the predetermined chromosome is at least one selected from chromosomes 18, 21 and 23.

57-63. (canceled)

64. A method for determining a chromosome aneuploidy of twins, comprising:
performing sequencing on cell-free nucleic acids contained in a peripheral blood sample obtained from a pregnant woman with twins, so as to obtain a sequencing result consisting of a plurality of sequencing data;
determining a fraction xᵢ of the number of sequencing data derived from chromosome i in the sequencing result to total sequencing data, where i represents a serial number of the chromosome, and i is any integer in the range of 1 to 22;
determining a T score of the chromosome i according to Tᵢ=(xᵢ−μᵢ)/σᵢ, where i represents the serial number of the chromosome and i is any integer in the range of 1 to 22, μᵢ represents an average percentage of sequencing data of the chromosome i selected as a reference system in a reference database to total sequencing data thereof, σᵢ represents a standard deviation of percentages of the sequencing data of the chromosome i selected as the reference system in the reference database to total sequencing data thereof,
determining an L score of the chromosome i according to Lᵢ=log(d(Tᵢ, a))/log(d(T2ᵢ, a)), where i represents the serial number of the chromosome and i is any integer in the range of 1 to 22, T2ᵢ=(xᵢ−μᵢ*(1+fra/2))/σᵢ; d(Tᵢ, a) and d(T2ᵢ, a) represent t distribution probability density function, where a represents degree of freedom, fra represents a first cell-free fetal DNA fraction determined by the method according to claim 1,
plotting a four-quadrant diagram with T as vertical coordinate and L as horizontal coordinate by zoning with a first straight line where T=predetermined fifth threshold and a second straight line where L=predetermined sixth threshold, wherein both fetuses of the twins are determined to have trisome if a sample under detection is determined to be of the T score and the L score falling into a first quadrant;
one fetus of the twins is determined to be of trisome and the other fetus of the twins is determined to be normal if a sample under detection is determined to be of the T score and the L score falling into a second quadrant;
both fetuses of the twins are determined to be noirual if a sample under detection is determined to be of the T score and the L score falling into a third quadrant;
the twins are determined to have a low fetal fraction if a sample under detection is determined to be of the T score and the L score falling into a fourth quadrant, such a result is not adopted.

65. A method for detecting fetal chimera, comprising:
performing sequencing on cell-free nucleic acids contained in a peripheral blood sample obtained from a pregnant woman with a fetus, optionally a male fetus, so as to obtain a sequencing result consisting of a plurality of sequencing data;
determining a first cell-free fetal DNA fraction, based on the sequencing data, by the method according to claim 1, or estimating a fetal fraction by chromosome Y (fra.chrY %) as a first cell-free fetal DNA fraction according to the following formula:
fra.chry=(chry.ER %−Female.chry.ER %)/(Man.chry.ER %−Female.chry.ER %)*100%,
where fra.chry chry.ER % represents the first cell-free fetal DNA fraction, represents a percentage of sequencing data derived from chromosome Y in the sequencing result to total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;
determining a third cell-free fetal DNA fraction based on sequencing data derived from a predetermined chromosome in the sequencing result; and
determining whether the fetus under detection has fetal chimera with respect to the predetermined chromosome based on the first cell-free fetal DNA fraction and the third cell-free fetal DNA fraction.

66. The method according to claim 65, wherein the third cell-free fetal DNA fraction is determined by the following formula:
fra.chri=2*(chri.ER %/adjust.chri.ER %−1)*100%,
where fra.chri represents the third cell-free fetal DNA fraction, i represents a serial number of the predetermined chromosome and i is any integer in the range of 1 to 22; chri.ER % represents a percentage of the sequencing data derived from the predetermined chromosome in the sequencing result to total sequencing data; adjust.chri.ER % represents an average percentage of sequencing data of cell-free nucleic acids derived from the predetermined chromosome in a peripheral blood sample obtained from a pregnant woman predetermined to be with a normal fetus to total sequencing data thereof.

67. The method according to claim 66, wherein determining whether the fetus under detection has fetal chimera with respect to the predetermined chromosome based on the first cell-free fetal DNA fraction and the third cell-free fetal DNA fraction further comprises:
(a) determining a ratio of the third cell-free fetal DNA fraction to the first cell-free fetal DNA fraction; and
(b) determining whether the fetus under detection has chimera with respect to the predetermined chromosome by comparing the ratio determined in (a) with a plurality of predetermined thresholds.

68. The method according to claim 67, wherein the plurality of predetermined thresholds comprises at least one selected from:
a seventh threshold, determined based on a plurality of control samples with the predetermined chromosome known to be of complete monosome,
an eighth threshold, determined based on a plurality of control samples with the predetermined chromosome known to be of monosome chimera,
a ninth threshold, determined based on a plurality of control samples with the predetermined chromosome known to be normal,
a tenth threshold, determined based on a plurality of control samples with the predetermined chromosome known to be of complete trisome,
optionally,
the predetermined chromosome of the fetus under detection is of complete monosome, if the ratio of the third cell-free fetal DNA fraction to the first cell-free fetal DNA fraction is lower than the seventh threshold;
the predetermined chromosome of the fetus under detection is of monosome chimera, if the ratio of the third cell-free fetal DNA fraction to the first cell-free fetal DNA fraction is not lower than the seventh threshold and not greater than the eighth threshold;
the predetermined chromosome of the fetus under detection is normal, if the ratio of the third cell-free fetal DNA fraction to the first cell-free fetal DNA fraction is greater than the eighth threshold and lower than the ninth threshold;
the predetermined chromosome of the fetus under detection is of trisome chimera, if the ratio of the third cell-free fetal DNA fraction to the first cell-free fetal DNA fraction is not lower than the ninth threshold and not greater than the tenth threshold; and
the predetermined chromosome of the fetus under detection is of complete trisome, if the ratio of the third cell-free fetal DNA fraction to the first cell-free fetal DNA fraction is greater than the tenth threshold.

69. The method according to claim 68, wherein the seventh threshold is greater than −1 and lower than 0, optionally is −0.85;
the eighth threshold is greater than the seventh threshold and lower than 0, optionally is −0.3;
the ninth threshold is greater than 0 and lower than 1, optionally is 0.3;
the tenth threshold is greater than the ninth threshold and lower than 1, optionally is 0.85.

70-74. (canceled)

75. A method for detecting fetal chimera, comprising:
performing sequencing on cell-free nucleic acids contained in a peripheral blood sample obtained from a pregnant woman with a fetus, so as to obtain a sequencing result consisting of a plurality of sequencing data;
determining a fraction xᵢ of the number of sequencing data derived from chromosome i in the sequencing result to total sequencing data, where i represents a serial number of the chromosome, and i is any integer in the range of 1 to 22;
determining a T score of the chromosome i according to Tᵢ=(xᵢ−μᵢ)/σᵢ, where i represents the serial number of the chromosome and i is any integer in the range of 1 to 22, μᵢ represents an average value of percentages of sequencing data of the chromosome i selected as a reference system in a reference database to total sequencing data thereof, σᵢ represents a standard deviation of percentages of the sequencing data of the chromosome i selected as the reference system in the reference database to total sequencing data thereof,
determining an L score of the chromosome i according to Lᵢ=log(d(Tᵢ,a))/log(d(T2ᵢ, a)) where i represents the serial number of the chromosome and i is any integer in the range of 1 to 22, T2ᵢ=(xᵢ−μᵢ*(1+fra/2))/σᵢ; d(Tᵢ, a) and d(T2ᵢ, a) represent t distribution probability density function, where a represents degree of freedom, fra represents a cell-free fetal DNA fraction determined by the method according to claim 1 or a fetal fraction estimated by chromosome Y (fra.chrY %),
fra.chry=(chry.ER %−Female.chry.ER %)/(Man.chry.ER %−Female.chry.ER %)*100%,
where fra.chry represents a cell-free fetal DNA fraction, chry.ER % represents a percentage of sequencing data derived from chromosome Y in the sequencing result to said total sequencing data; Female.chry.ER % represents an average percentage of sequencing data of cell-free nucleic acids derived from chromosome Y in a peripheral blood sample obtained from a pregnant woman predetermined to be with a normal female fetus to total sequencing data thereof; and Man.chry.ER % represents an average percentage of sequencing data of cell-free nucleic acids derived from chromosome Y in a peripheral blood sample obtained from a healthy man predetermined to total sequencing data thereof;
plotting a four-quadrant diagram with T as vertical coordinate and L as horizontal coordinate by zoning with a third straight line where T=predetermined eleventh threshold and a fourth straight line where L=predetermined twelfth threshold, when the T score is greater than 0,
wherein the fetus is determined to have complete monosome or monosome chimera with respect to the predetermined chromosome, if a sample under detection is determined to be of the T score and the L score falling into a first quadrant;
the fetus is determined to have monosome chimera with respect to the predetermined chromosome, if a sample under detection is determined to be of the T score and the L score falling into a second quadrant;
the fetus is determined to be normal with respect to the predetermined chromosome, if a sample under detection is determined to be of the T score and the L score falling into a third quadrant;
the fetus is determined to have a low fetal fraction if a sample under detection is determined to be of the T score and the L score falling into a fourth quadrant, such a result is not adopted,
plotting a four-quadrant diagram with T as vertical coordinate and L as horizontal coordinate by zoning with a first straight line where T=predetermined thirteenth threshold and a second straight line where L=predetermined fourteenth threshold, when the T score is greater than 0,
wherein the fetus is determined to have complete trisome or trisome chimera with respect to the predetermined chromosome, if a sample under detection is determined to be of the T score and the L score falling into a first quadrant;
the fetus is determined to have trisome chimera with respect to the predetermined chromosome, if a sample under detection is determined to be of the T score and the L score falling into a second quadrant;
the fetus is determined to be normal with respect to the predetermined chromosome, if a sample under detection is determined to be of the T score and the L score falling into a third quadrant;
the fetus is determined to have a low fetal fraction if a sample under detection is determined to be of the T score and the L score falling into a fourth quadrant, such a result is not adopted,
optionally, the eleventh threshold and the thirteenth threshold each independently is 3, and the twelfth threshold and the fourteenth threshold each independently is 1.

76. (canceled)

77. The method according to claim 14, wherein the predetermined function is d=0.0334*p+1.6657, where d represents a fraction of cell-free fetal nucleic acids, and p represents a percentage of cell-free nucleic acid present in the predetermined range.
